(19) United States
(12) Patent Application Publication
(10) Pub. No.: US 2024/0057880 A1
(43) Pub. Date: Feb. 22, 2024

(54) PROCESS AND SIGNAL PROCESSING UNIT FOR DETERMINING A CARDIOGENIC REFERENCE SIGNAL SEGMENT

(71) Applicant: Drägerwerk AG & Co. KGaA, Lübeck (DE)

(72) Inventor: Lorenz KAHL, Lübeck (DE)

(21) Appl. No.: 18/365,281

(22) Filed: Aug. 4, 2023

(30) Foreign Application Priority Data
Aug. 18, 2022 (DE) 10 2022 120 871.0

(51) Int. Cl. A61B 5/024; A61B 5/0205; A61B 5/00; G16H 40/67; G16H 50/30

(52) U.S. Cl. CPC A61B 5/024; A61B 5/0205; A61B 5/7221; A61B 5/7246; G16H 40/67; G16H 50/30; A61B 5/0816

(57) ABSTRACT

A process and a signal processing unit generate a cardiogenic reference signal segment (SigA_{kar,ref}) describing patient cardiac activity during the course of a heartbeat. A sum signal (Sig_{Sum}) is generated that includes a superposition of a respiratory signal with a cardiogenic signal. A sample is generated including one sum signal segment [SigA_{Sum}(x₁), . . . , SigA_{Sum}(x_{N})] per heartbeat for a sequence of heartbeats. For each heartbeat, a characteristic heartbeat time point [H_Zp(x₁), . . . , H_Zp(x_{N})] is detected. The cardiogenic reference signal segment is calculated by aggregating the sum signal segments and using the heartbeat time points. For aggregation, a weight factor (w1, w2, . . . ) is used for each sum signal segment. The weight factor depends on how well the sum signal and related segments have been generated, the detection reliability of the heartbeat time points, and/or an evaluation of the shape of the sum signal segment or the generated cardiogenic reference signal segment.

### CROSS REFERENCE TO RELATED APPLICATIONS

This application claims the benefit of priority under 35 U.S.C. § 119 of German Application 10 2022 120 871.0, filed Aug. 18, 2022, the entire contents of which are incorporated herein by reference.

### TECHNICAL FIELD

The invention relates to a process and a signal processing unit which are capable of determining by computation, and thereby generating, a cardiogenic reference signal segment and which use for this purpose a sample with measured values from a patient. The cardiogenic reference signal segment describes at least approximately the cardiac activity of the patient in the course of a single heartbeat. The cardiac activity is superimposed on the patient's own breathing activity. A patient's “own breathing activity” is understood to be the breathing activity performed by the patient with his or her own respiratory muscles, in particular due to spontaneous breathing and/or optional external stimulation of the patient's own respiratory muscles.

### BACKGROUND

One possible application of the invention is to determine, at least approximately, a respiratory signal. The respiratory signal describes a patient's own breathing activity. The respiratory signal usually cannot be measured directly. Rather, it is determined using a sum signal, wherein the sum signal is measured and comprises a superposition of the sought respiratory signal with a cardiogenic signal, and wherein the cardiogenic signal describes the patient's cardiac activity. In order to determine the respiratory signal, the influence of the cardiogenic signal on the sum signal is at least approximately compensated (eliminated) by computation.

Another possible application of the invention is that at least approximately a cardiogenic signal is determined. The cardiogenic signal describes the cardiac activity of the patient. The cardiogenic signal can also generally not be measured directly, but only determined approximately. For this purpose, the cardiogenic signal is composed of cardiogenic signal segments, each cardiogenic signal segment describing at least approximately the cardiac activity of the patient in the course of a single heartbeat and calculated using the invention.

### SUMMARY

It is an object of the invention to provide a process and a signal processing unit which are able to calculate a cardiogenic reference signal segment more reliably than known processes and signal processing units.

The task is solved by a process having process features according to the invention and by a signal processing unit having signal processing unit features according to the invention and by a system having system features according to the invention. Advantageous embodiments of the process according to the invention are, as far as useful, also advantageous embodiments of the signal processing unit and the system according to the invention and vice versa. Preferably, the process according to the invention is carried out using the signal processing unit or the system according to the invention.

The process and the signal processing unit according to the invention automatically provide a cardiogenic reference signal segment. This cardiogenic reference signal segment describes the cardiac activity of a patient in the course of a single heartbeat, preferably an average cardiac activity.

The process according to the invention comprises the following steps, which are performed automatically, and the signal processing unit according to the invention is adapted to automatically perform the following steps:
A sensor arrangement measures in and/or on and/or at the patient's body at least one variable, preferably an anthropological variable. Preferably, the measured variable or each measured variable correlates with the cardiac activity and/or the patient's own breathing activity, particularly preferably with a superposition of these two activities, optionally with further activities or other signals which are performed in or on the patient's body and/or act on the patient's body from outside.
The sensor arrangement provides measured values. The process according to the invention is carried out using measured values from the sensor arrangement, preferably processed measured values. The signal processing unit according to the invention is configured to receive and process measured values from the sensor arrangement. The sensor arrangement is not necessarily a part of the signal processing unit.
A sum signal is generated. This sum signal comprises a superposition of a respiratory signal with a cardiogenic signal. Optionally, the sum signal comprises at least one signal from a further source. The respiratory signal describes the patient's own breathing activity. The cardiogenic signal describes the patient's cardiac activity. To generate the sum signal, measured values of the sensor arrangement are used, and a measured value pre-processing is applied to these measured values.
A sample is generated. This sample comprises for a sample sequence of heartbeats one sum signal segment per heartbeat of the sample sequence. Each sum signal segment for a heartbeat of the sample sequence describes the course of the sum signal in the course of this heartbeat.
For each heartbeat of the sample sequence, a characteristic heartbeat time point is detected. Preferably, the sum signal segment for this heartbeat is evaluated for this purpose.
The cardiogenic reference signal segment is calculated using an aggregation of the sum signal segments of the sample sequence, preferably as such an aggregation.
To calculate the cardiogenic reference signal segment by aggregation, the characteristic heartbeat time points are further used, in particular to position the sum signal segments of the sample in correct time (temporally correct) on a time axis.

To calculate the aggregation, for each sum signal segment of the sample at least one respective weight factor is calculated and used. Preferably, a weighted average of the sum signal segments of the sample is calculated as the aggregation.

The weight factor or at least one weight factor for a sum signal segment, preferably each weight factor for a sum signal segment, is calculated depending on at least one respective quality measure. At least one of the following three quality measures is used, optionally at least two quality measures are used:
first quality measure: a quality measure for the quality with which the sum signal and/or the sum signal segment for the heartbeat have been generated by pre-processing a measured value,
second quality measure: a quality measure of the reliability with which the characteristic heartbeat time points (heartbeat timing) of the heartbeat has been detected,
third quality measure: a quality measure that assesses (evaluates) a form of the sum signal segment for that heartbeat and/or the generated cardiogenic reference signal segment.

In one embodiment, the second quality measure is used as well as the first quality measure and/or the third quality measure.

The larger the used quality measure is, the greater is the weight factor or any weight factor produced using at least one of these quality measures. Note: This applies if the greater the quality measure, the greater the quality, i.e. the better the respective result. Conversely, if the greater the quality, the smaller the quality measure, then the smaller the quality measure used, the greater the weight factor.

In one embodiment, a weighting is calculated for each sum signal segment, for which at least one of the quality measures just mentioned is used, optionally at least two quality measures. If the used quality measure is lower than a given threshold, the weight factor zero is used for this sum signal segment. In other words, a sum signal segment with a low quality will not be considered. If the quality measure is above the lower threshold, the weight factor is calculated as a function of the quality measure such that the larger the quality measure, the larger the weight factor. For example, the quality measure is used as the weight factor.

According to the invention, a characteristic heartbeat time point is detected for a heartbeat of the sample sequence. The time course of a human heartbeat has a characteristic course and typically has five peaks, namely a P-peak to a T-peak (P QRS T). In particular, the characteristic heartbeat time point is one of these peaks or a characteristic time point between two of these peaks, e.g., a time average.

According to the invention, a cardiogenic reference signal segment is generated. This cardiogenic reference signal segment describes the cardiac activity of a patient in the course of a single heartbeat, i.e. it is a section of a cardiogenic signal, whereby this section refers to a heartbeat period.

The cardiogenic reference signal segment generated according to the invention can be used to approximate the contribution of a single heartbeat to the sum signal. In particular, the cardiogenic reference signal segment can be used to computationally compensate for the contribution of this heartbeat to the sum signal and thereby obtain a respiratory signal. The cardiogenic reference signal segment as well as detected heartbeat time points can also be used to generate a cardiogenic signal. In this case, the cardiogenic reference signal segment is applied several times, namely once for each heartbeat considered. The cardiogenic reference signal segments are positioned in correct time, and the characteristic heartbeat time points are used for the correct time positioning. The heartbeat time points can be determined using the sum signal.

As a rule, it is not possible to measure the cardiogenic signal or the respiratory signal directly. However, it is possible to generate the sum signal using measured values from the sensor arrangement. The sum signal comprises a superposition of the cardiogenic signal with the respiratory signal. In particular, when the sum signal is an electrical signal, the contribution that the cardiogenic signal makes to the sum signal in the course of a heartbeat is significantly greater than the contribution of the respiratory signal. Therefore, the sum signal can be used to detect the sum signal segments for the heartbeats. Furthermore, these sum signal segments can be used to detect the characteristic heartbeat time points. In an intermediate time span between two immediately successive heartbeats, however, the sum signal is often determined exclusively or predominantly by the respiratory signal, so that the segments of the sum signal in these intermediate time spans are essentially characterized by the respiratory signal and can therefore be determined in the sum signal.

According to the invention, a sample with several sum signal segments is generated for the heartbeats of the sample sequence. This sample is automatically generated from the sum signal, so that no further measured values are required, but only those used for the generation of the sum signal. The sum signal is measured on the patient to whom the cardiogenic reference signal segment to be generated and optionally the respiratory signal to be determined also refers. Therefore, the sample is also obtained on this patient.

The cardiogenic reference signal segment is calculated by aggregating the sum signal segments of the sample. Because a sample is used, it is possible, but not necessary thanks to the invention, to specify a model assumption about the cardiogenic reference signal segment. Furthermore, the cardiogenic reference signal segment is calculated for a patient using a sample obtained by means of measured values from that patient. As a result, it is not necessary to use measured values or signals that are used for multiple patients and therefore necessarily only approximate the cardiac activity of a particular patient. Further, it is not necessary to use an average cardiac activity that is valid for multiple patients. The cardiac activity of an individual patient often deviates greatly from an average cardiac activity.

It would be conceivable to calculate the cardiogenic reference signal segment by calculating the arithmetic mean or a median or other averaging over the sum signal segments of the sample. However, with this approach, individual outliers could significantly influence the result and thereby distort it. An outlier is a sum signal segment with a clearly deviating course. Such an outlier can be caused in particular by the following events:
In one implementation, the sensor arrangement includes multiple measurement electrodes positioned on the patient's skin that generate measurements non-invasively, i.e., without the need to insert an instrument into the patient's body to take measurements. In this implementation, an outlier can be caused by a measurement electrode being moved relative to the patient's skin or losing contact with the skin. For example, the patient or other human touches or moves the measurement electrode. It is also possible that touching the skin changes an electrical potential and thus falsifies a measured value.
It is also possible that the sensor arrangement comprises a sensor positioned in the patient's body, for example a probe in the esophagus. If the patient moves or processes in the patient's body change the position or the effect of the sensor, an outlier may occur.
Individual sum signal segments may be affected by an interfering signal, such as electromagnetic radiation emanating from an electrical device or a power supply network in the vicinity of the sensor arrangement.
The patient's cardiac activity shows individual significant irregularities, for example, because the patient is physically exerting himself or herself or is agitated, or he or she is being medically examined or administered a drug.

According to the invention, at least one weight factor per heartbeat and thus per sum signal segment is used in each case during aggregation. This weighting factor depends on at least one quality measure. The greater the quality measure, i.e. the greater the quality of the delivered result, the greater the weight factor. Thanks to this feature, outliers have less influence on the cardiogenic reference signal segment than if an averaging were performed, in particular an averaging in which all sum signal segments are included in the cardiogenic reference signal segment to the same extent.

The invention makes it possible, but avoids the need, to check by another sensor whether one of the causes of an outlier described above is actually present. Furthermore, thanks to the invention, it is not necessary to determine and/or disregard time periods in which an outlier is present. Rather, outliers usually result in at least one significantly reduced quality measure, and the corresponding sum signal segment is therefore less influential in the cardiogenic reference signal segment thanks to the weight factors according to the invention.

As explained above, a sum signal is generated from measured values of the sensor arrangement, wherein the sum signal comprises a superposition of a cardiogenic signal with a respiratory signal. In one application of the invention, the respiratory signal is determined. For this purpose, the contribution of the cardiogenic signal to the sum signal is computationally compensated. In order to compensate this contribution computationally, the cardiogenic reference signal segment generated according to the invention is used.

In an advanced embodiment of this application, in order to detect the respiratory signal, the cardiogenic reference signal segment is computationally multiplied so that one copy is generated for each heartbeat considered. Furthermore, for each heartbeat considered, the respective characteristic heartbeat time is detected, preferably by evaluating the sum signal. The copies of the cardiogenic reference signal segments are positioned in correct time relative to the sum signal using the characteristic heartbeat time points. Subsequently, the correctly timed positioned cardiogenic reference signal segments are subtracted from the sum signal. The difference is an estimate for the respiratory signal (also referred to as a representation of the respiratory signal).

In another application of the invention, the cardiogenic signal is determined. Again, the cardiogenic reference signal segment is computationally duplicated, and for a sequence of heartbeats, the respective characteristic heartbeat timing is detected. The copies of the cardiogenic reference signal segments are positioned in correct time on a time axis, for which the characteristic heartbeat times are used. Gaps between two consecutive copies of the cardiogenic reference signal segment are filled, for example by interpolation. The result is an estimate for the cardiogenic signal (also referred to as a representation of the cardiogenic signal).

According to the invention, a sample with N sum signal segments is used to generate the cardiogenic reference signal segment. Preferably, the process according to the invention is repeated continuously. Particularly preferably, a sample with the last N heartbeats in each case is used in each repetition. It is possible to continuously change the last cardiogenic reference signal segment obtained, for which the sample with the sum signal segments of the last N heartbeats is used. Thanks to the continuous repetition, the process automatically adapts to a possible change in the patient's cardiac activity. This change in cardiac activity does not necessarily need to be measured directly. This embodiment saves computing time compared to an embodiment in which the cardiogenic reference signal segment is calculated again from zero each time.

According to the invention, a cardiogenic reference signal segment is generated which describes the cardiogenic signal in the course of a single heartbeat. Further above, two applications were described in which copies of the cardiogenic reference signal segment were generated, namely one copy for each heartbeat of a sequence. In addition, the characteristic heartbeat timing (time points) of the heartbeats of the sequence are detected. The copies are positioned with correct timing.

In one modification, an adapted cardiogenic signal segment for a heartbeat is generated from each copy of the cardiogenic reference signal segment for that heartbeat. This modification can be used both to determine the respiratory signal and to determine the cardiogenic signal. For this adaptation, it is measured what value a given anthropological parameter takes for the patient during this heartbeat. Preferably, this measurement is performed again for each heartbeat. The anthropological parameter is, for example, a position of the patient, in particular in a patient bed, or the current filling level of his or her lungs, or the time interval between two immediately successive heartbeats, or even a disturbance signal acting on the patient from outside. The value of the anthropological parameter may change from heartbeat to heartbeat. The adjusted cardiogenic signal segments are again positioned at the correct time, for which the characteristic heartbeat time points are used.

In one application, the signal processing unit according to the invention and the sensor arrangement are components of a system which artificially ventilates a patient and thereby supports the patient's own breathing activity. A patient-side coupling unit is positioned in and/or on the body of a patient, for example a breathing mask or a tube or a catheter. The system further comprises a ventilator that performs a sequence of ventilatory breaths and delivers an amount of a gas to the patient-side coupling unit in each ventilatory breath. The gas includes oxygen and optionally at least one anesthetic. Using the invention as described above, the signal processing unit calculates an estimate (representation) for the respiratory signal. This estimate is used to synchronize the ventilator breaths with the patient's own breathing activity. Synchronization refers at least to synchronization in time, and ideally also synchronization in terms of amplitude (strength).

In the following, the invention is described by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a graph showing an exemplary section (segment) of a cardiogenic signal in the course of a single heartbeat;

FIG. 2 is a schematic view showing various sensors that measure different quantities used to determine a respiratory signal representation;

FIG. 3 is a graph showing an exemplary course of the sum signal as well as exemplary two heartbeat times (time points) and two heartbeat periods;

FIG. 4 is a schematic view showing several functional blocks for determining the estimated respiratory signal, where no signal qualities are considered;

FIG. 5 is a view with graphs to exemplify how a cardiogenic reference signal segment is generated and used under ideal conditions;

FIG. 6 is a view with graphs showing an exemplary course of the compensation signal and two exemplary heartbeat periods;

FIG. 7 is a view with graphs showing the signal waveforms of FIG. 5, with external disturbances affecting the cardiogenic reference signal segment;

FIG. 8 is a schematic view showing the function blocks of FIG. 4 and additional function blocks which consider signal qualities;

FIG. 9 is a view with graphs showing the signal waveforms of FIG. 7, taking signal qualities into account;

FIG. 10 is a view showing how a time course of an overall quality measure is used for each heartbeat;

FIG. 11 is graph showing an example of a computation of a baseline;

FIG. 12 is a view with graphs showing the raw signal, two baselines calculated in different ways, the sum signal and a quality measure;

FIG. 13 is a schematic view showing the measured value conditioner and the function block that calculates a quality measure for the measured value conditioner; and

FIG. 14 is a schematic view showing the function block that calculates a quality measure for the detection of the characteristic heartbeat time points (timing).

### DESCRIPTION OF PREFERRED EMBODIMENTS

Referring to the drawings, in an embodiment, the invention is used for artificial ventilation and/or automatic evaluation of vital parameters of a patient.

In the following, a “signal” is to be understood as the course in the time domain or also in the frequency domain of a directly or indirectly measurable and time-varying variable which correlates with a physical variable. In the present case, this physical variable is related to the cardiac activity and/or the patient's own breathing activity and/or other muscular activity and/or to the artificial ventilation of the patient and is generated by at least one signal source in the patient's body and/or externally by a respirator. A “respiratory signal” correlates with the patient's own breathing activity, and a “cardiogenic signal” correlates with the patient's cardiac activity. A section of this signal that relates to a specific time period is referred to below as a “signal segment”. The value of a signal at a certain point in time is called the signal value or also the signal segment value.

In the embodiment example, the invention is used to automatically determine an estimate Sig_{res,est} for a respiratory signal Sigᵣₑₛ, wherein the respiratory signal to be estimated Sigᵣₑₛ correlates with the patient's own breathing activity of a patient P and therefore describes his or her own breathing activity. This patient's own breathing activity can be triggered by electrical impulses in the body of the patient P, whereby the patient P generates these impulses himself or herself, i.e. a spontaneous breathing, and/or being stimulated from the outside, for example in a magnetic field. The index est indicates that the respiratory signal is estimated (is a representation) and not measured exactly.

In both cases, the patient P's diaphragm muscles perform this patient's own breathing activity. This distinguishes the patient's own breathing activity from artificial ventilation, which is caused by ventilation strokes of a ventilator. Artificial ventilation can replace the patient's own breathing activity, especially if the patient is completely anesthetized (mandatory artificial ventilation), or supplement the patient's own breathing activity (supportive artificial ventilation).

In one application of the embodiment, the patient P is at least temporarily artificially ventilated by supportive artificial ventilation while the estimated respiratory signal Sig_{res,est} is determined. In another application, the invention is used to monitor the patient P and, in particular, the patient P's own breathing activity and to use the respiratory signal Sigᵣₑₛ to be estimated for this purpose, without the patient P necessarily being artificially ventilated continuously.

Both the respiratory signal Sigᵣₑₛ and the calculated estimate Sig_{res,est} are time-varying, i.e., Sigᵣₑₛ=Sigᵣₑₛ(t) and Sig_{res,est}=Sig_{res,est}(t).

This respiratory signal Sigᵣₑₛ cannot be measured directly. It is possible to position a measuring probe in the body of patient P and to generate measured values from the probe. It is also possible to obtain measured values by non-invasive means, in particular by having electrodes record measured values on the skin of the patient P. In general, it is not possible by either invasive or non-invasive means to directly measure the pulses generated in the patient P's body that “drive” the respiratory muscles, but only electrical readings generated when the respiratory muscle fibers contract, or the effects of such electrical readings. Moreover, the electrical impulses which cause patient P's own breathing activity are superimposed by electrical impulses which cause patient P's cardiac activity, more precisely: which are generated when the cardiac muscles contract. Therefore, after appropriate processing of measured values, only a sum signal Sig_{Sum} can be measured directly. This sum signal Sig_{Sum} results from a superposition of the sought respiratory signal Sigᵣₑₛ, which correlates with patient P's own breathing activity, and a cardiogenic signal Sigₖₐᵣ, which correlates with cardiac activity. The summed signal Sig_{Sum} can be influenced by other signals, in particular by those signals acting on a transmission channel from the signal source to the measurement location, as well as by external signal sources.

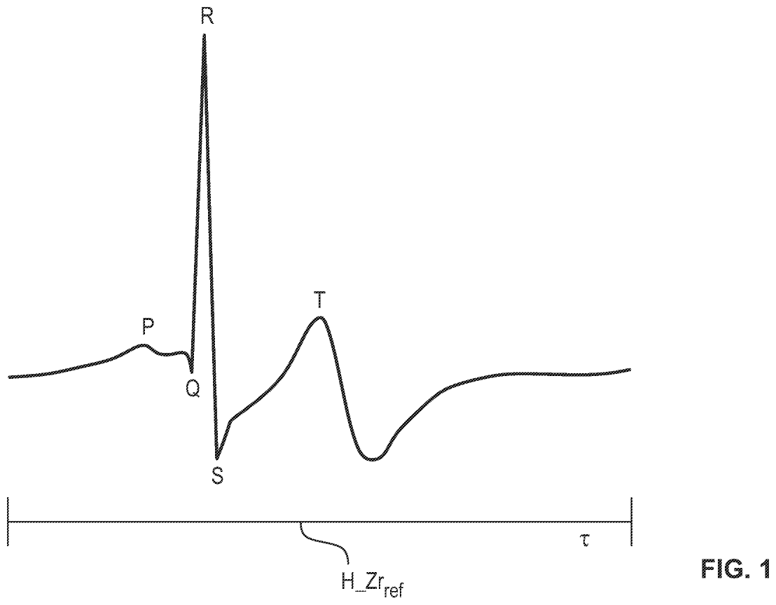

FIG. 1 shows a typical section of an electrically measured cardiogenic signal Sigₖₐᵣ in the course of a single heartbeat. A reference heartbeat period H_Zr_{ref} is shown on the x-axis, and the signal value, for example in millivolts, is shown on the y-axis. Five peaks P, Q, R, S and T are shown. The characteristic heartbeat time is for example the Q-peak, the R-peak, the S-peak or also the time midpoint between the Q-peak and the S-peak of this heartbeat or the time midpoint between the P-peak and the T-peak.

The sum signal Sig_{Sum} arises from the superposition of the respiratory signal Sigᵣₑₛ with the cardiogenic signal Sigₖₐᵣ. As a rule, the sum signal Sig_{Sum} is additionally superimposed by interfering signals.

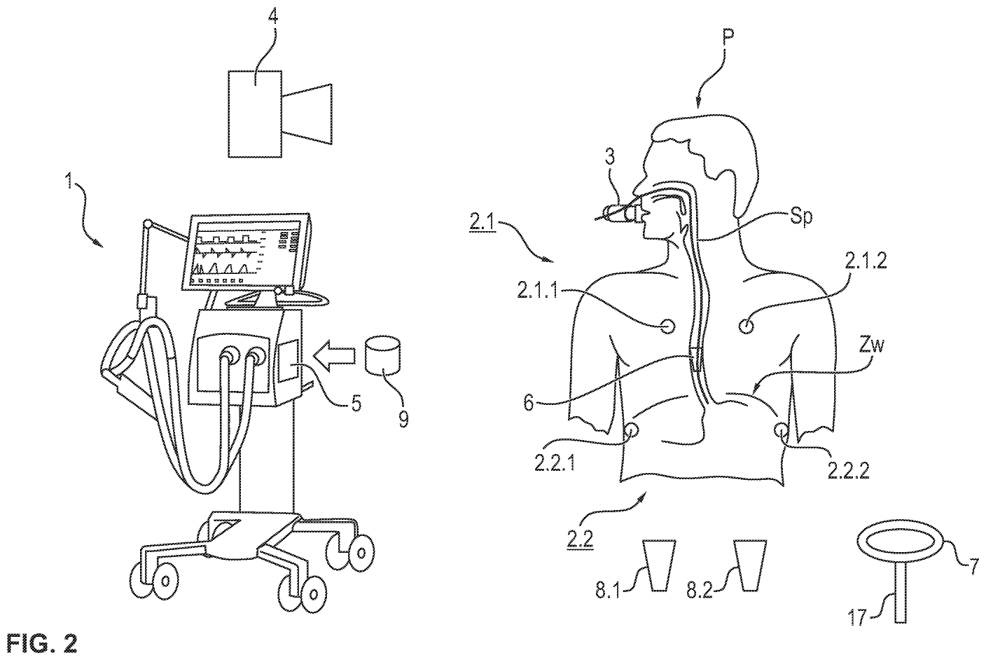

FIG. 2 schematically shows which signals can be generated from measured values by automatically processing the measured values in a suitable manner. Schematically shown are
the artificially ventilated patient P,
the esophagus Sp and the diaphragm Zw of the patient P,
a ventilator 1 which artificially ventilates the patient P at least intermittently and which comprises a processing signal processing unit 5, the signal processing unit 5 having at least intermittent read access and write access to a data memory 9,
an intercostal pair 2.1 with two measuring electrodes 2.1.1 and 2.1.2, which are arranged to the right and left of the sternum and between each two ribs of patient P, i.e. in an area close to the heart,
a pair 2.2 close to the diaphragm with two measuring electrodes 2.2.1 and 2.2.2 located close to the diaphragm of patient P,
an electrode not shown for ground,
a pneumatic sensor 3, which is spatially remote from the body of the patient P and comprises a measurement transducer arranged, for example, in front of the mouth of the patient P, and a data-processing evaluation unit, which may be arranged in the ventilator 1,
an optional optical sensor 4, comprising an image acquisition device and an image evaluation unit, directed at the body of the patient P,
an optional pneumatic sensor 6 in the form of a probe or balloon in the esophagus Sp and near the diaphragm Zw of the patient P,
a schematically shown cuff 7 around a wrist of the patient P, said cuff 7 holding a catheter 17 to invasively measure the time course of blood pressure,
two finger clips 8.1, 8.2, each placed over a finger of patient P or positioned elsewhere on the skin of patient P, one finger clip 8.1 measuring non-invasively the degree of saturation of the blood with oxygen, preferably by means of a plethysmographic process, and the other finger clip 8.2 measuring non-invasively the blood pressure of patient P, and
optionally not shown electrodes in the esophagus of patient P.

The intercostal pair 2.1 and the ground electrode provide a first sum signal Sig_{Sum}(1) after signal conditioning. The pair 2.2 near the diaphragm and the ground electrode provide a second sum signal Sig_{Sum}(2) after signal conditioning. The other sensors described above can supply further sum signals Sig_{Sum}(n), n>=3. It is also possible for the same sensor arrangement to supply two different sum signals, for example by using different measurement processes. Such a sensor arrangement is described, for example, in DE 10 2009 035 018 A1 (corresponding US2011028819 (A1) is incorporated by reference). In the following, we will refer to “the sum signal Sig_{Sum}” for short.

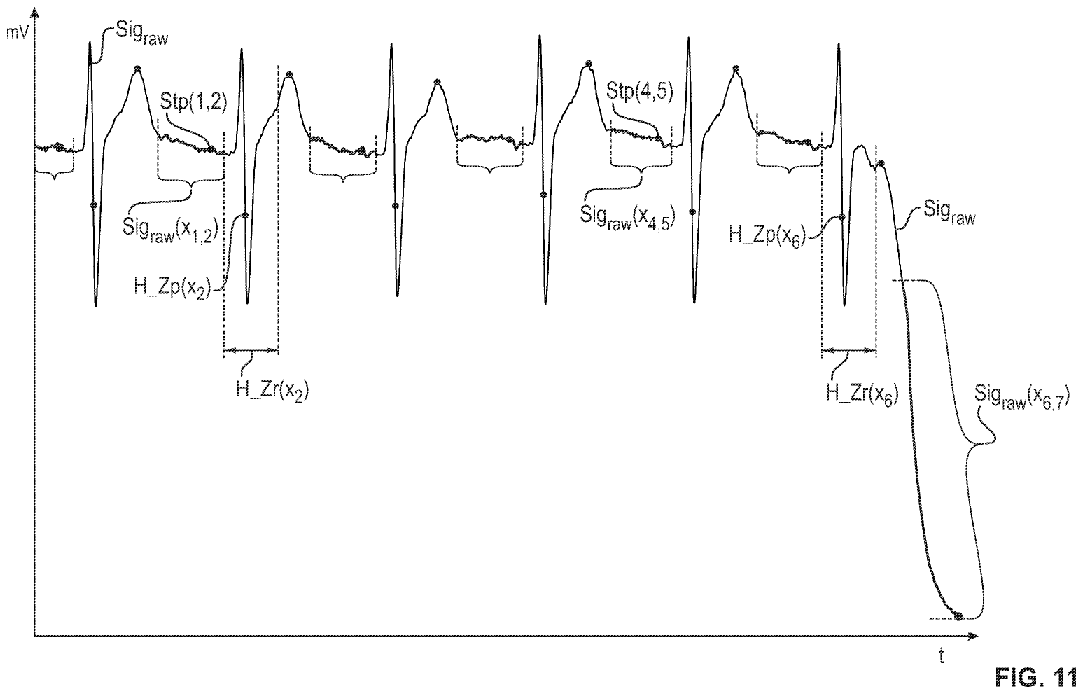

Preferably, the signal processing comprises a so-called baseline filtering. This is described in more detail below with reference to FIG. 11.

Instead of an electrical signal (EMG signal), a sum signal Sig_{Sum} in the form of a mechanomyogram (MMG signal) can also be generated and used, for example. For the embodiment example, only the EMG or MMG sensors are required. It is also possible to generate as a sum signal Sig_{Sum} such a signal that correlates with the time course of the change in blood volume in the patient's body P, for example with the aid of measured values obtained by optical plethysmography.

The optical sensor 4 repeatedly and contactlessly measures in each case a value for at least one time-varying anthropological parameter of the patient P. The parameter is, for example, the current lung filling level and/or the current sitting posture of the patient P or the time interval between two immediately successive heartbeats (interval between two successive R peaks). The optical sensor 4 comprises, for example, a camera or other image acquisition unit and an image evaluation unit.

From the measured values of the other sensors and of sensors not shown inside the ventilator 1, a measure P_{aw} for the respiratory pressure and/or a measure Pₑₛ for the esophageal pressure can be generated, and from this a pneumatic measure Pₘᵤₛ can be derived, which is also a measure of the patient P's own breathing activity. According to a preferred implementation, on the one hand an estimate Sig_{res,est} for the electrical or mechanical respiratory signal Sigᵣₑₛ and on the other hand a pneumatic measure Pₘᵤₛ are determined. Thanks to this combination, the patient's own breathing activity P is determined with higher reliability than when deriving and using only one signal. Furthermore, thanks to this combination, it is possible to derive in many cases how well the respiratory muscles of patient P convert electrical impulses in patient P's body into pneumatic breathing activity (neuromechanical efficiency). The invention can also be used in an embodiment in which the EMG signal or the MMG signal is generated, but not the pneumatic measure Pₘᵤₛ of breathing activity.

The estimated respiratory signal Sig_{res,est} determined according to the invention is used, for example, for the following purposes:
A pneumatic measure Pₘᵤₛ for patient P's own breathing activity is derived using the respiratory signal Sig_{res,est}. The ventilatory strokes effected by the ventilator 1 are synchronized as well as possible with the patient P's own breathing activity, synchronized in time and preferably also in terms of amplitude (strength). Ideally, a ventilatory stroke begins when the patient P starts his or her own inspiratory effort. The ventilation stroke ends when patient P completes this inspiratory effort.
The neuromechanical efficiency of patient P's breathing is determined. This efficiency is a measure of how well the respiratory muscles convert electrical signals into spontaneous breathing.
The condition of the patient's respiratory muscles P is determined (fatigue determination)—the pneumatic measure Pₘᵤₛ is not required for this purpose,
Asynchronies in the patient's own breathing activity P are detected—again, the pneumatic measure Pₘᵤₛ is not needed,
To monitor vital signs of the patient P, the estimated respiratory signal Sig_{res,est} and the respiratory EMG power are determined and output as two vital signs in a form perceivable by a human, preferably visually in the form of a time course each, optionally together with the airway pressure P_{aw} and/or the esophageal pressure Pₑₛ,
It is possible that patient P is not fully sedated, but that the diaphragm muscles of patient P perform their own breathing activity. In this situation, depending on the estimated respiratory signal Sig_{res,est}, support of the patient's own breathing activity by artificial ventilation is triggered and/or performed. Preferably, the ventilatory strokes of the artificial ventilation are performed depending on the estimated respiratory signal Sig_{res,est}. For example, the ventilator 1 triggers and/or terminates the ventilation strokes depending on the estimated respiratory signal Sig_{res,est} and/or determines the respective amplitude of each ventilation stroke and/or the time-varying frequency of the ventilation strokes depending on the estimated respiratory signal Sig_{res,est}. The amplitude, duration and end of a ventilation stroke can also be controlled automatically depending on the estimated respiratory signal Sig_{res,est}.

In order to control the ventilator 1 during artificial ventilation of the patient P or in order to monitor the patient P and to use the estimated respiratory signal Sig_{res,est} for the control or monitoring, the estimated respiratory signal Sig_{res,est} is determined with a high sampling frequency, i.e. at each sampling time t the signal processing unit 5 supplies a new signal value Sig_{res,est}(t). By a “high sampling frequency” it is understood that there is an interval of less than five, preferably less than three milliseconds between two successive sampling times. In particular, for fatigue determination, the sampling frequency is preferably at least 1 kHz, more preferably at least 2 kHz. In contrast, some steps of the process described below are carried out in the embodiment example with a low sampling frequency, namely with a frequency that lies in the range of the heartbeat frequency, i.e. between 1 Hz and 2 Hz.

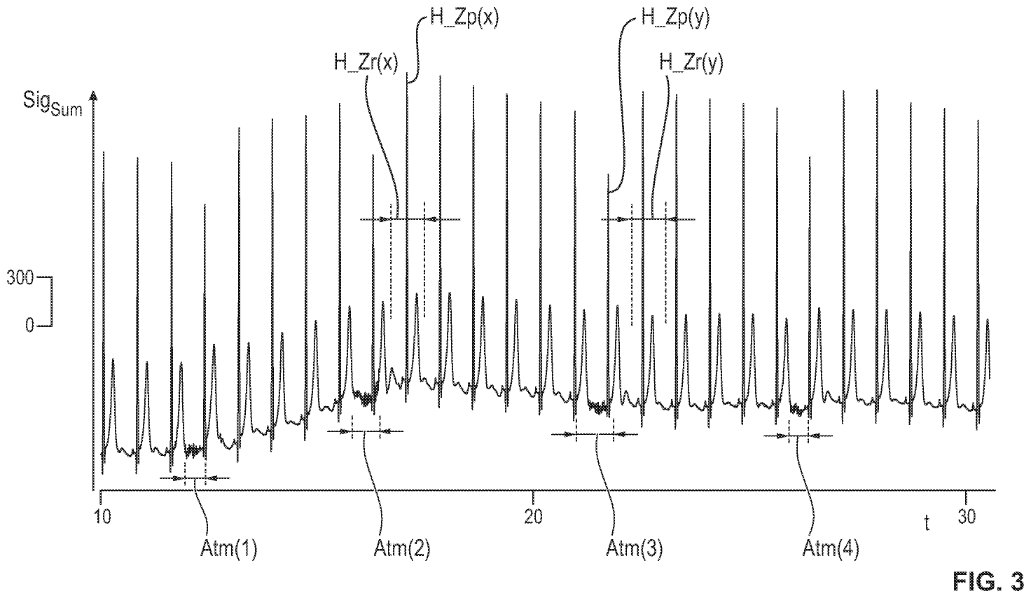

FIG. 3 shows an exemplary time course of a sum signal Sig_{Sum}. The segment shown in FIG. 3 comprises four breaths and a plurality of heartbeats. Shown are the four time periods Atm(1), . . . , Atm(4) of the four breaths and for the two exemplary heartbeats x and y respectively a heartbeat time period H_Zr(x) and H_Zr(y) and a characteristic heartbeat time point H_Zp(x) and H_Zp(y). It can be seen that the cardiogenic signal Sigₖₐᵣ in a heartbeat period is several times larger than the respiratory signal Signs in this period. However, outside of a heartbeat period H_Zr(x), H_Zr(y), the respiratory signal Sigᵣₑₛ is sufficiently strong compared to the cardiogenic signal Sigₖₐᵣ and therefore can be determined from the sum signal Sig_{Sum}, for example, by using the sum signal Sig_{Sum} as the estimated respiratory signal Sig_{res,est} outside of each heartbeat period H_Zr(x), H_Zr(y).

The sum signal Sig_{Sum} or each sum signal Sig_{Sum} is a superposition of the sought respiratory signal Sigᵣₑₛ and the cardiogenic signal Sigₖₐᵣ and optionally interfering signals. In one application of the invention, the characteristic heartbeat time point H_Zp(x) of each heartbeat x is used to computationally compensate for the influence of the cardiogenic signal Sigₖₐᵣ on a sum signal Sig_{Sum}.

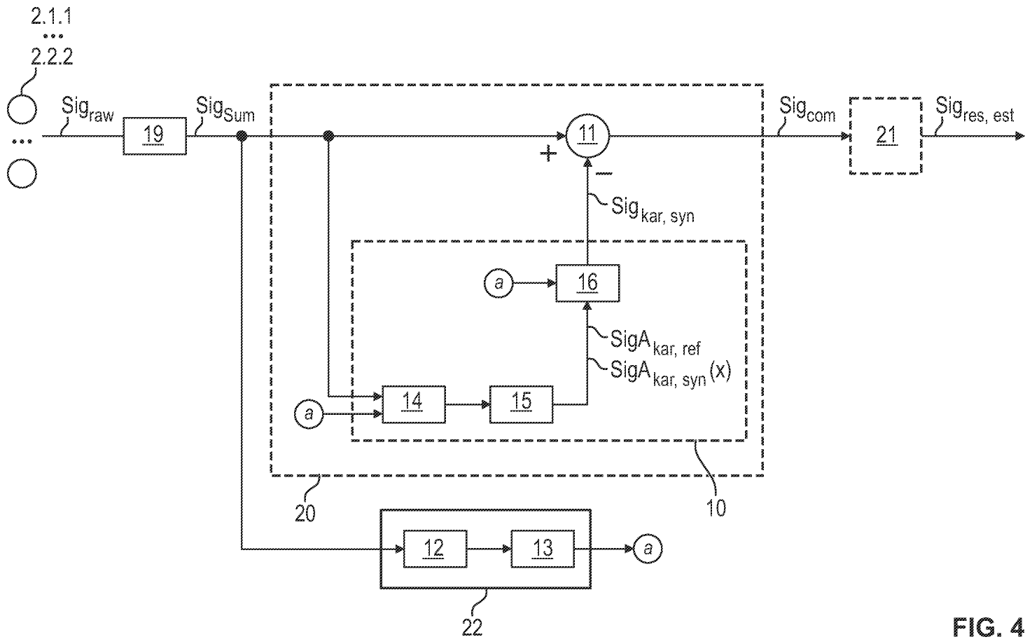

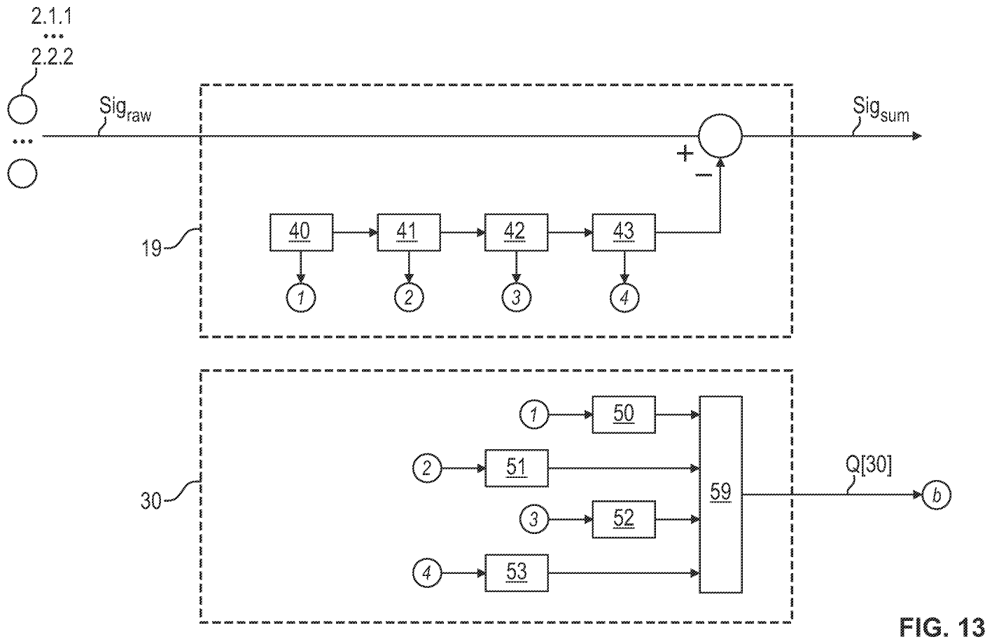

FIG. 4 schematically shows a measured value conditioner 19. This measured value conditioner 19 conditions the raw signal Sig_{raw} which is supplied by the sensors 2.1.1 to 2.2.2 after signal amplification. The measured value conditioner 19 removes low-frequency oscillations by computation, normalizes the raw signal Sig_{raw} and supplies the sum signal Sig_{Sum}. This is described in more detail below with reference to FIG. 13.

FIG. 4 further shows schematically a function block 20 which receives the sum signal Sig_{Sum} and performs the just mentioned computational compensation of the cardiogenic signal Sigₖₐᵣ. This function block 20 performs various signal processing steps in order to eliminate by computation the contribution of the cardiogenic signal Sigₖₐᵣ in a sum signal Sig_{Sum}, i.e. to at least partially computationally compensate for the influence of cardiac activity on a measured sum signal Sig_{Sum}. Thus, an approximation Sig_{res,est} for the sought respiratory signal Sigᵣₑₛ is obtained. For each heartbeat x, the function block 20 detects in the sum signal Sig_{Sum} a sum signal segment SigA_{Sum}(x) in which the heartbeat x occurs. After an appropriate temporal transformation, each sum signal segment SigA_{Sum}(x) relates to the same reference heartbeat time period H_Zr_{ref}, cf. FIG. 1. In the heartbeat time period H_Zr(x), the sum signal Sig_{Sum} is determined almost exclusively by the cardiogenic signal Sigₖₐᵣ, so that the other signal components can be neglected. The function block 20 provides a compensation signal Sig_{com} as a result.

A functional unit 10 of the compensation function block 20 generates a synthetic cardiogenic signal Sig_{kar,syn}, which is an approximation (estimate) for, particularly a representation of, the cardiogenic signal Sigₖₐᵣ and is composed of signal segments. The compensation function block 11 computationally compensates for the contribution of the cardiogenic signal Sigₖₐᵣ to m sum signal Sig_{Sum}, for example by subtracting the synthetic cardiogenic signal Sig_{kar,syn}, thereby generating the compensation signal Sig_{com}. Exemplary procedures to generate such a compensation signal Sig_{com} are described in
In M. Ungureanu and W. M. Wolf, “Basic Aspects Concerning the Event-Synchronous Interference Canceller,” IEEE Transactions on Biomedical Engineering, Vol. 53 No. 11 (2006), pp. 2240-2247 (the publication is incorporated by reference),
In L. Kahl and U. G. Hofmann: “Removal of ECG artifacts from EMG signals with different artifact magnitudes by template subtraction,” Current Directions in Biomedical Engineering 2019; 5(1), pp. 357-360 (the publication is incorporated by reference),
in DE 10 2007 062 214 B3 (corresponding U.S. Pat. No. 8,109,269 (B2) is incorporated by reference),
in EP 3 381 354 A1 (corresponding U.S. Ser. No. 11/596,340 (B2) is incorporated by reference), and
in DE 10 2019 006 866 A1 (corresponding US2022330837 (A1) is incorporated by reference).

In one embodiment, the compensation function block 20 applies one of the procedures described therein.

In an initialization phase Ip, the compensation function block 20 generates a cardiogenic reference signal segment SigA_{kar,ref} which is valid for this patient P in this current situation and which is stored in the data memory 9, and applies this cardiogenic reference signal segment SigA_{kar,ref} again in a subsequent use phase Np for each heartbeat. Preferably, the initialization phase Ip is repeated continuously, for which the respective last N heartbeats are used. In this way, the reference signal segment SigA_{kar,ref} is continuously updated and, in particular, adapts to a changed state of patient P. Preferably, N is between 50 and 100. In some figures, the value N=9 is used for simplification to keep the illustration clear.

The following steps are performed in both phases Ip, Np:
A functional unit 12 identifies in the sum signal Sig_{Sum} the heartbeat period H_Zr(x) of each heartbeat x, preferably the respective start, e.g. the P peak, and the respective end, e.g. the T peak, and/or the respective QRS phase of each heartbeat x, cf. FIG. 1.
A functional unit 13 determines the respective characteristic heartbeat time point H_Zp(x) of each heartbeat x, preferably with a tolerance of a few milliseconds. Particularly preferably, the tolerance is at most half the time span between two successive sampling times for determining the sum signal Sig_{Sum}, with this time span preferably being less than 1 millisecond.
The function block 22 with the functional units 12 and 13 requires several values of the sum signal Sig_{Sum} for several successive sampling times in order to determine the characteristic heartbeat time point H_Zp(x), H_Zp(y) of each heartbeat x, y with sufficient accuracy. What consequences this has, for example, for the control of the ventilator 1 is explained further below.

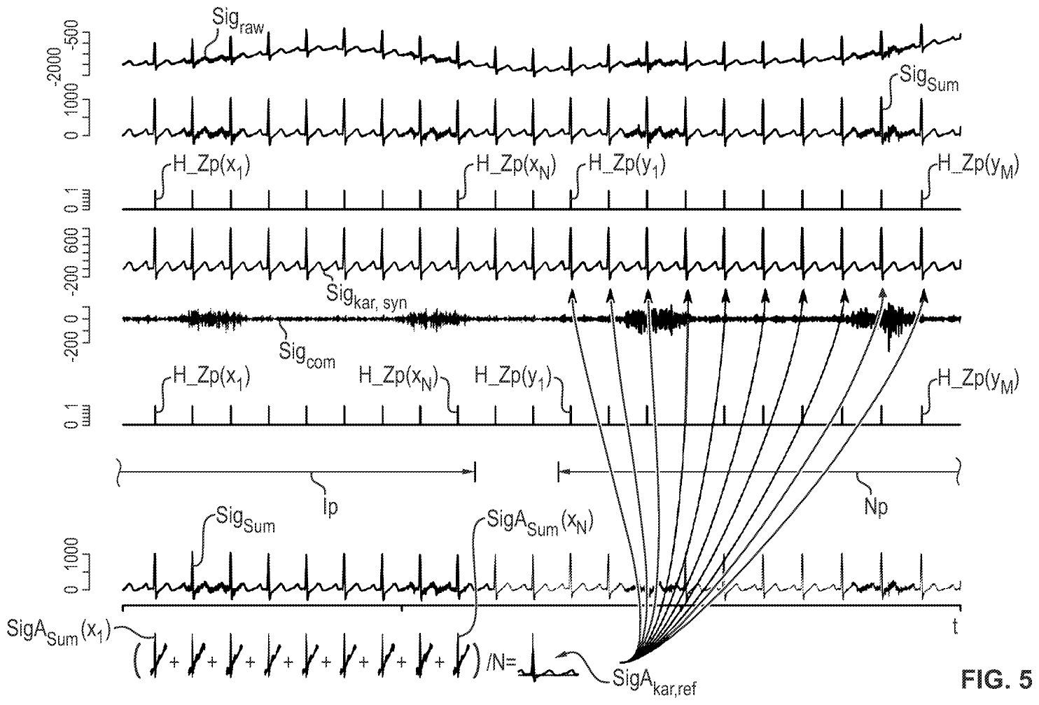

In the initialization phase Ip, the following steps continue to be performed:
A sum signal segment SigA_{Sum}(x) of the sum signal Sig_{Sum}, cf. FIG. 3 and FIG. 5, belongs to each heartbeat number x. A functional unit 14 computationally superimposes the N sum signal segments SigA(x_{Sum1}), . . . , SigA_{Sum}(x)_{N} for the last N heartbeats x₁, . . . , x_{N}. If necessary, these N sum signal segments SigA_{Sum}(x₁), . . . , SigA_{Sum}(x_{N}) are trimmed or compressed or stretched to a matching length and also mapped to the same reference heartbeat period H_Zr_{ref}. A procedure for superimposing sections is described in M. Ungureanu and W. M. Wolf, op. cit.
Preferably, the N sum signal segments SigA_{Sum}(x₁), . . . , SigA_{Sum}(x_{N}) are superimposed for the N heartbeats such that the N sum signal segments SigA_{Sum}(x₁), . . . , SigA_{Sum}(x_{N}) have the same length and the R peaks are superimposed. Each sum signal segment SigA_{Sum}(x) thus refers to the same reference heartbeat period H_Zr_{ref}. A relative time point in this reference heartbeat period H_Zr_{ref} is denoted by τ. To each absolute time point t of the sum signal segment SigA_{Sum}(x) there corresponds a relative time point τ=τ(t) in this relative heartbeat time period. Instead of the designation “relative time point”, the designation “heart phase ϕ” with a value range from 0° to 360° or from 0 to 2π can also be used.
A functional unit 15 generates a cardiogenic reference signal segment (template) SigA_{kar,ref} from the superposition of N sum signal segments SigA_{Sum}(x₁), . . . , SigA_{Sum}(x_{N}), which the functional unit 14 has generated. This cardiogenic reference signal segment SigA_{kar,ref} approximately describes the course of the cardiogenic signal Sigₖₐᵣ during a single heartbeat and also relates to the reference heartbeat period H_Zr_{ref}. Preferably, the characteristic heartbeat time H_Zp(x) is at τ=0. As mentioned above, during a heartbeat the cardiogenic component in the sum signal Sig_{Sum} is many times larger than the respiratory component, and by averaging over N sum signal segments the respiratory components during a heartbeat are largely “averaged out”. The functional unit 15 preferably applies a learning procedure to the N sum signal segments SigA_{Sum}(x₁), . . . , SigA_{Sum}(x)_{N} for the last N heartbeats. The cardiogenic reference signal segment SigA_{kar,ref} is preferably stored in the data memory 9.

In the use phase Np, the following steps are performed:
The functional unit 13 detects the heartbeats in the sum signal Sig_{Sum} and determines the respective characteristic heartbeat time H_Zp(x) of each heartbeat x detected.
For each heartbeat x the cardiogenic reference signal segment SigA_{kar,ref} is used again. In one embodiment, this is subtracted unchanged from the sum signal segment SigA_{kar,ref} (template subtraction). This step is performed by the functional unit 16.
Optionally, on the other hand, the functional unit 16 additionally uses the respective value of at least one anthropological parameter which influences the cardiac activity and thus the cardiogenic signal Sigₖₐᵣ and which has been measured during this heartbeat no. x. The lung filling level LF and a measure of the current posture of the patient P as well as the distance RR between the R-peaks of two successive heartbeats are examples of such an anthropological parameter. For each heartbeat, the functional unit 16 adjusts the cardiogenic reference signal segment SigA_{kar,ref} to the parameter or to each parameter value of the anthropological parameter or each anthropological parameter measured at that heartbeat x, thereby generating an adjusted cardiogenic signal segment SigAₖₐᵣ(x) for heartbeat x.
The functional unit 16 positions the cardiogenic reference signal segment SigA_{kar,ref} or optionally the adapted cardiogenic signal segment SigAₖₐᵣ(x) relative to the sum signal segment SigA_{Sum}(x) of the current heartbeat in correct time, e.g. QRS synchronized. This generates a new synchronized section SigA_{kar,syn}(x) of the synthetic cardiogenic signal Sig_{kar,syn}. Preferably, the synthetic cardiogenic signal Sig_{kar,syn} is output in a form that can be perceived by a human.
A functional unit 11 compensates for the influence of the cardiogenic signal Sigₖₐᵣ in the latest sum signal segment SigA_{Sum}(x), for example by subtracting the cardiogenic reference signal segment SigA_{kar,ref} or the adjusted cardiogenic signal segment SigAₖₐᵣ(x) from the latest sum signal segment SigA_{Sum}(x).

A preferred embodiment for this, to apply a learning process in the initialization phase Ip and the respective value of an anthropological parameter for each heartbeat in the utilization phase, is described in the German disclosure DE 10 2019 006 866 A1 (discussed above).

At the beginning of the procedure, i.e. after the patient P is connected to the measuring electrodes 2.1.1 to 2.2.2, the initialization phase Ip is carried out, which covers a period of N heartbeats. Preferably, this initialization phase Ip is carried out again, namely with the last N heartbeats in each case. In this initialization phase Ip, the compensation function block 20 generates, as described above, depending on the sum signal segments SigA_{Sum}(x₁), . . . , SigA_{Sum}(x_{N}) for the last N heartbeats, an initial cardiogenic reference signal segment SigA_{kar,ref}.

During the procedure, i.e. in the use phase Np, the compensation function block 20 adapts the cardiogenic reference signal segment SigA_{kar,ref} to the respective last N heartbeats, i.e. to the last N sum signal segments SigA_{Sum}(x₁), . . . , SigA_{Sum}(x_{N}), and stores it in the data memory 9. The steps in the initialization phase Ip and the adaptation to the respective last N heartbeats are performed with the low sampling frequency, which is approximately equal to the heartbeat frequency.

Preferably, the N sum signal segments for each are superimposed with twice the time resolution of the sum signal Sig_{Sum}. This means: the values of the sum signal Sig_{Sum} are determined with a high sampling frequency f, i.e. the distance Δt between two sampling times is 1/f. It is possible to measure with the sampling frequency f. It is also possible to measure with a lower sampling frequency than f and to increase the frequency computationally. Computationally, the time resolution is increased to for even to e.g. 2f or 3f, e.g. by computationally positioning a signal value Sig_{Sum}(t+Δt/2) between two signal values Sig_{Sum}(t) and Sig_{Sum}(t+Δt) derived from measured values, for example by interpolation. The step of compensating for the influence of the cardiogenic signal Sigₖₐᵣ is preferably performed at the high sampling frequency f.

After the initialization phase Ip, the following steps are performed with the high sampling frequency (a few milliseconds or even only a few tenths of a millisecond):
The signal processing unit 5 derives a new value Sig_{Sum}(t) for the sum signal Sig_{Sum} from each of the measured values.
The function block 22 with the functional units 12 and 13 detects the start or the exact characteristic time H_Zp(x) of a heartbeat x in the sum signal Sig_{Sum} and thereby determines a new sum signal segment SigA_{Sum}(x).
Optionally, the compensation function block 20 adjusts the cardiogenic reference signal segment SigA_{kar,ref} to the respective value of at least one anthropological parameter, determines the associated relative time τ=τ(t), and generates another signal segment, namely the temporally most recent section SigA_{kar,syn}(x) of the synthetic cardiogenic signal Sig_{kar,syn}, by positioning it correctly in time.
The functional unit 11 subtracts from the new value Sig_{Sum}(t) the value SigA_{kar,ref}[τ(t)] or SigA_{kar,syn}(x) [τ(t)] of the cardiogenic reference signal segment SigA_{kar,ref} or the value of the adapted cardiogenic signal segment SigA_{kar,syn}(x) for the same relative time τ, i.e.

Sig_{com}(t)=Sig_{Sum}(t)−SigAₖₐᵣ[τ(t)] or

Sig_{com}(t)=Sig_{Sum}(t)−SigA_{kar,syn}(x)[τ(t)].
It is also possible that the functional unit 11 compensates for the cardiogenic influence in another way, for example with the aid of a high-pass filter or by means of an independent component analysis or by means of a so-called blind source separation, which is described, for example, in DE 10 2015 015 296 A1 (corresponding U.S. Ser. No. 11/202,605 (B2) is incorporated by reference).
The compensation function block 20 outputs a new signal segment SigA_{com}(x) for the compensation signal Sig_{com}.

In one embodiment, the output signal Sig_{com} of the compensation function block 20 is used as the estimated signal Sig_{res,est} for the sought respiratory signal Sigᵣₑₛ. In another embodiment, the output signal is attenuated, by an attenuation function block 21, cf. FIG. 4. Preferably, the attenuation function block 21 comprises a high-pass filter with a cutoff frequency between 10 Hz and 50 Hz to remove low-frequency residuals of the cardiogenic signal Sigₖₐᵣ. An exemplary realization of this attenuation function block 21 is described in the German disclosure DE 10 2020 002 572 A1 (corresponding US2021338176 (A1) is incorporated by reference).

FIG. 5 shows an example of how a cardiogenic reference signal segment SigA_{kar,ref} is generated. The x-axis shows the time in [sec], where the distance between two bars is 5 sec, and the y-axis shows the respective signal value in μN. Also shown are the initialization phase Ip and the use phase Np. In the example shown, N=9.

The following signals are shown in FIG. 5 from top to bottom:
the raw signal Sig_{raw},
the sum signal Sig_{Sum}, which the measured value conditioner 19 generates from the raw signal Sig_{raw} and which is shown in the second row from above and in the second row from below,
The sequence of detected heartbeat time points H_Zp(x₁), . . . , H_Zp(x_{N}), H_Zp(y₁), . . . , H_Zp(y_{M}), . . . .
the synthetic cardiogenic signal Sig_{kar,syn},
the compensation signal Sig_{com}, which is generated from the cardiogenic reference signal segment SigA_{kar,ref} using the detected characteristic heartbeat times H_Zp(y₁), . . . , H_Zp(y_{M}), . . . ,
again the sequence of detected heartbeat time points H_Zp(x₁), . . . , H_Zp(x_{N}), H_Zp(y₁), . . . ,
the N sum signal segments SigA_{Sum}(x₁), . . . , SigA_{Sum}(x_{N}) for the N heartbeat periods H_Zr(x₁), . . . , H_Zr(x_{N}) of the initialization phase Ip,
the sum signal Sig_{Sum},
schematically, how the arithmetic mean is formed over the N sum signal segments SigA_{Sum}(x₁), . . . , SigA_{Sum}(x_{N}) and used as the cardiogenic reference signal segment SigA_{kar,ref}.

It can be seen that the raw signal Sig_{raw} has low-frequency oscillations, i.e. oscillation with a frequency lower than the heartbeat frequency. In addition, the signal values are between −2000 μV and −500 μN. The sum signal Sig_{Sum} has signal values between 0 μV and 1000 μV and no longer exhibits low-frequency oscillations because these have been eliminated by computation.

In the subsequent use phase Np, the compensation signal Sig_{com} is used using the cardiogenic reference signal segment SigA_{kar,ref}, and the heartbeat time H_Zp(y1), . . . H_Zp(y_{M}), . . . is calculated. As already stated, preferably the cardiogenic reference signal segment SigA_{kar,ref} is continuously updated, for which the respective last N sum signal segments SigA_{Sum}(x₁), . . . , SigA_{Sum}(x_{N}) are used.

The upper four signals are generated in such a way that a shorter processing time is realized and thus a higher specified real-time requirement is fulfilled. The “lead time” is understood to be the time interval between the generation of the measured values used for the signal and the generation of the respective signal. The lower three signals are generated with a longer lead time, i.e. a lower specified real-time requirement, and therefore usually with higher accuracy and/or reliability.

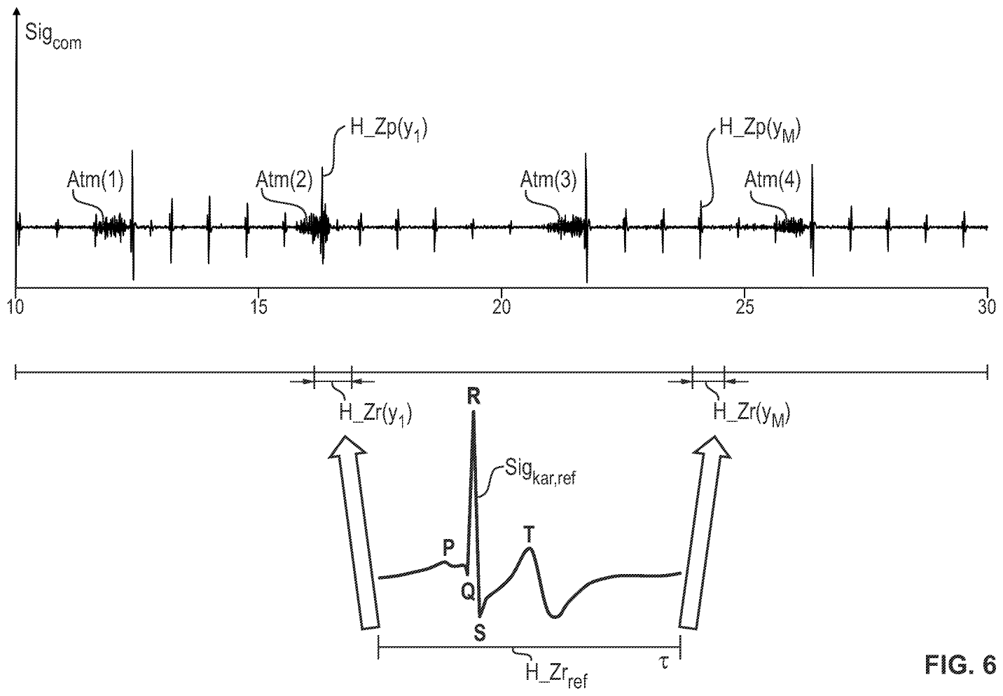

FIG. 6 shows above an exemplary course of the compensation signal Sig_{com}. This exemplary course results from the fact that the compensation function block 20 processes the sum signal Sig_{Sum} exemplarily shown in FIG. 4 and FIG. 5 as just described. Also shown in FIG. 6 are four time periods Atm(1), . . . , Atm(4) of patient P's own breathing activities and two exemplary characteristic heartbeat time points H_Zp(y₁) and H_Zp(y_{M}) as well as the two corresponding heartbeat time periods H_Zr(y₁) and H_Zr(y_{M}).

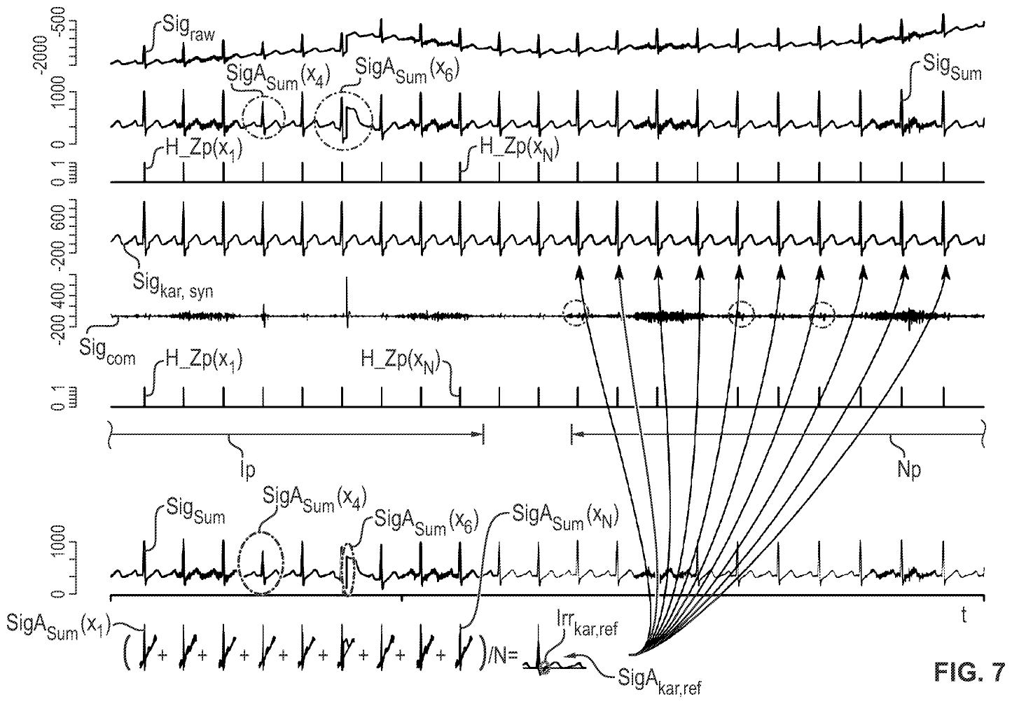

The signal curves in FIG. 5 result from ideal conditions. FIG. 7 shows the signal curves of FIG. 5, where various external disturbances act on the measuring electrodes 2.1.1, . . . , 2.2.2, the signal processing unit 5 and/or on the patient P and/or where the heart activity of the patient P is irregular. One cause of external interference is that a measuring electrode 2.2.1 to 2.2.2 is not properly attached to the body of patient P or is moved or even temporarily loses contact with the skin. In addition, a disturbing electrical radiation can occur. Identical reference signs have the same meanings in FIG. 7 as in FIG. 5.

In FIG. 7, the following differences from FIG. 5 are visible:
The sum signal segment SigA_{Sum}(x₄) for the heartbeat x₄ turned out unusually low and was also distorted.
The sum signal segment SigA_{Sum}(x₆) for the heartbeat x₆ also shows an unusual time course after the S-peak, in particular a jump.

The unusual time course of the sum signal segment SigA_{Sum}(x₆) for the heartbeat x₆ can have the following causes in particular:
The patient P himself or herself or another person touches a measuring electrode 2.2.1 to 2.2.2, which changes the contact to the skin of the patient P. The contact to the skin of the patient P changes.
A measuring electrode 2.2.1 to 2.2.2 loses contact with the skin, so that the signal is not a measure of resistance but of capacitance.
A measuring electrode 2.2.1 to 2.2.2 moves relative to the body of the patient P, for example because someone touches the measuring electrode 2.2.1 to 2.2.2.

These irregularities lead to a deviating course Irr_{kar,ref} as part of the cardiogenic reference signal segment SigA_{kar,ref}. This deviant course causes the compensation signal Sig_{Com} to deviate significantly from zero even outside a breath period Atm(1), . . . . This does not correspond to anthropological reality. In the following, a remedy for this problem according to the invention is described as an example.

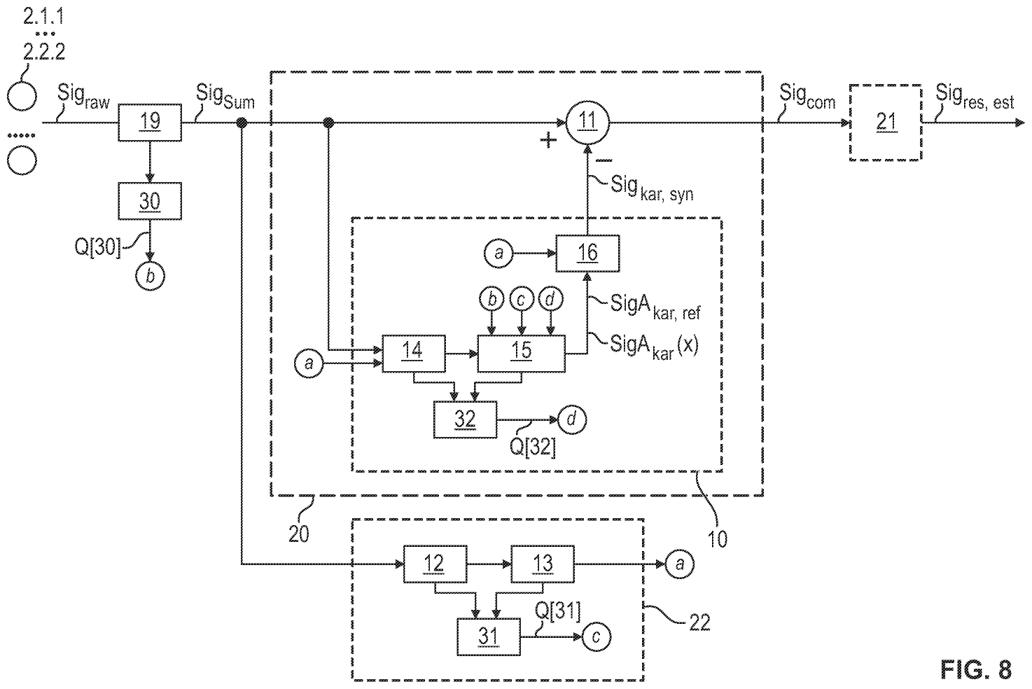

FIG. 8 shows an extension of the functional circuit diagram of FIG. 4 according to the invention. The same reference signs have the same meanings as in FIG. 4. The following additional function blocks are shown:
A function block 30 evaluates the quality with which the function block 19 has generated the sum signal Sig_{Sum} from the measured values of the sensors 2.1.1 to 2.2.2. Function block 30 supplies a quality measure Q[30], which comprises one value for each heartbeat x.
A function block 31 evaluates how regular the heartbeat x is and with what reliability the characteristic heartbeat time H_Zp(x) was detected by evaluating the sum signal Sig_{Sum}. Function block 31 provides a quality measure Q[31] that comprises one value for each heartbeat x.
A function block 32 evaluates whether the determined cardiogenic reference signal segment SigA_{kar,ref} or the adapted cardiogenic signal segment SigAₖₐᵣ(x) for a heartbeat x matches predefined expectations of a cardiogenic signal segment. For this purpose, in particular, it is evaluated whether the matched cardiogenic signal segment SigAₖₐᵣ(x) has a time course with Q to T as shown in FIG. 1. The function block 32 provides a quality measure Q[32]. In one embodiment, the quality measure Q[32] comprises a single value for each heartbeat x, in another embodiment a time course of values.

It is possible that the heart activity of the patient P acts on at least two different sum signals, in particular on sum signals from different sensors. In one embodiment, different heartbeat times are detected. However, they all originate from the same heart and are therefore different estimates (representations) for the same event. In one embodiment, a heartbeat time point is selected from a signal. In one embodiment, the function block 31 evaluates how much the estimates for a heartbeat time differ from each other and calculates the quality measure Q[31] depending on the differences.

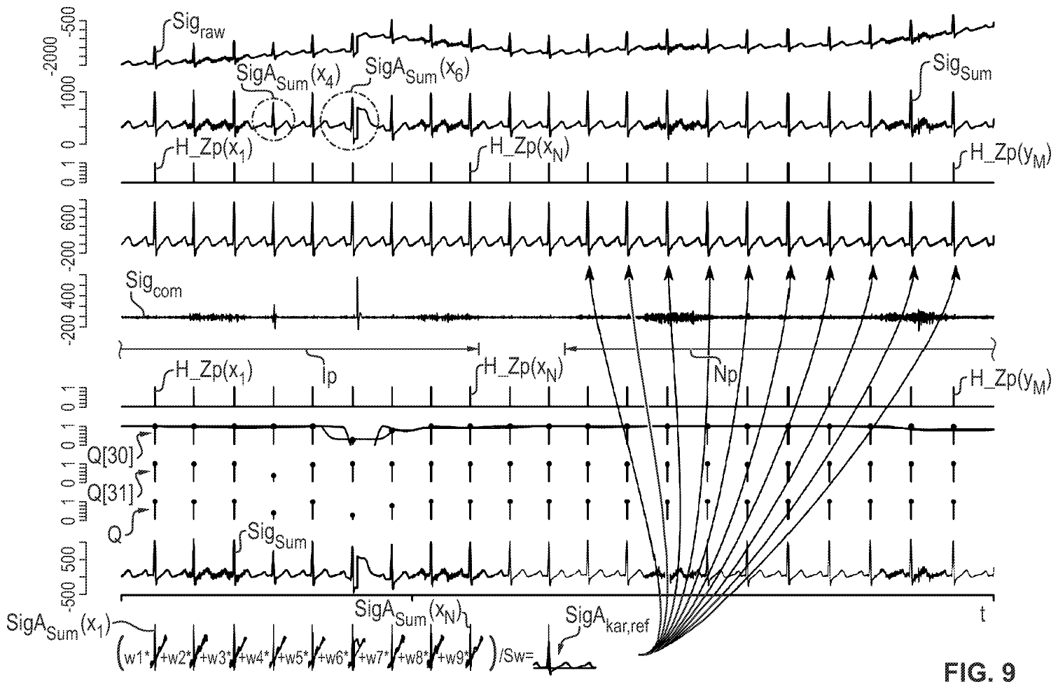

FIG. 9 shows the signal curves of FIG. 7. In this example, the two quality measures Q[30] and Q[31] determined by the two function blocks 30 and 31 are additionally shown, namely one value per heartbeat in each case, where this value lies between 0 and 1 inclusive and is plotted in one measurement series in each case. It is possible to additionally consider the quality measure Q[32] of function block 32.

All three quality measures Q[30], Q[31], Q[32] are the greater, the greater the respective quality. From the quality measures Q[30], Q[31] and/or Q[32], an overall quality measure Q is derived, which in each case comprises one value per heartbeat x and which is also plotted in a measurement series in FIG. 9. For example, for each heartbeat the product or the maximum or the minimum of the values of Q[30], Q[31] and/or Q[32] is formed.

A weight factor per heartbeat is derived from the total quality measure Q, i.e. in the example shown nine weight factors w1, . . . , w9 for the N=9 heartbeats of the initialization phase Ip. The larger the total quality measure Q for a heartbeat, the larger the weight factor for this heartbeat. Sw is used to denote the sum w1+ . . . +w9. In the example shown,

SigA_{kar,ref}=[w1*SigA_{Sum}(x₁)+wN*SigA_{Sum}(x_{N})]/Sw.

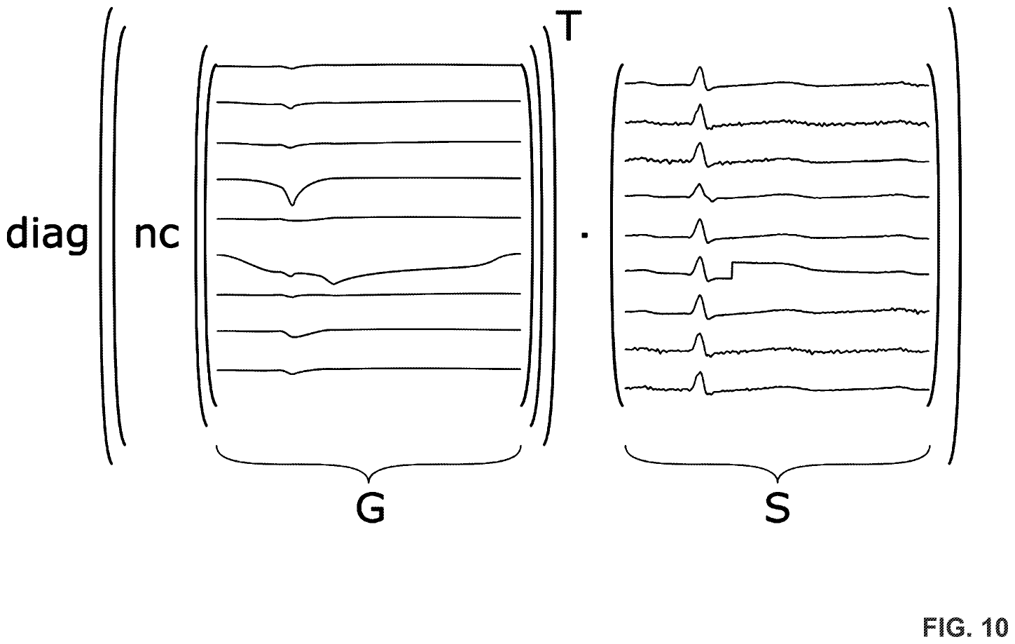

FIG. 10 illustrates an embodiment in which not a single value but a time course of the total quality measure Q is calculated and used for each heartbeat. Again, N=9 heartbeats are used. In a heartbeat period H_Zr(x₁), . . . , H_Zr(x_{N}) there are M sampling times each. The matrix G has one row per heartbeat and one column per sampling time, so it is a matrix with N rows and M columns. Each row of the matrix G shows the time course of the total quality measure Q over the course of a single heartbeat. G^{T} is the transposed matrix, which has M rows and N columns. The matrix S also has one row per heartbeat and one column per sampling time. Each row of matrix S shows the sum signal segment SigA_{Sum}(x), SigA_{Sum}(y) of the sum signal for heartbeat x and y, respectively. The matrix [nc(G)]^{T}×S is calculated. In the matrix nc(G), the sum of the values in each column is normalized to one. [nc(G)]^{T} is the transpose of nc(G). The square matrix product [nc(G)]^{T}×S has M rows and M columns. The vector diag {[nc(G)]^{T}×S} consists of the M values of the main diagonal of the matrix [nc(G)]^{T}×S. This main diagonal provides a time course and is used as the cardiogenic reference signal segment SigA_{kar,ref}.

FIG. 13 shows an example of several functional units of the measured value conditioner 19 as well as a function block 30, which evaluates the quality Q[30] with which the measured value conditioner 19 generates the sum signal Sig_{Sum} from the raw signal Sig_{raw}.

The measurement conditioner 19 subtracts from the raw signal Sig_{raw} a kind of average curve (baseline) BL, see FIG. 5, FIG. 7 and FIG. 9.

FIG. 11 illustrates a preferred arrangement for calculating the average curve (baseline) BL. Shown is a segment of the raw signal Sig_{raw}, which comprises the six heartbeats x₁, . . . , x₆. A sequence of segments Sig_{raw}(x_{n,n+1}), Sig_{raw}(x_{n+1,n+2}), . . . of the raw signal Sig_{raw} between each two consecutive heartbeat periods H_Zr(xₙ) and H_Zr(xₙ₊₁), H_Zr(xₙ₊₁) and H_Zr(xₙ₊₂), is determined. Thus, the section Sig_{raw}(x_{1,2}) lies between the two heartbeat periods H_Zr(x₁) and H_Zr(x₂), and so on. Preferably, a predetermined time interval of Δt each occurs between the period covered by the section Sig_{raw}(x_{n,n+1}) and the two adjacent heartbeat periods H_Zr(xₙ) and H_Zr(xₙ₊₁).

For each section Sig_{raw}(x_{1,2}), Sig_{raw}(x_{2,3}), a support point Stp(1,2), Stp(2,3), . . . is determined. A spline is drawn through this sequence of support points. The section of the spline between two neighboring support points is a polynomial. Preferably, a Piecewise Cubic Hermite Interpolating Polynomial is used as the spline, where a third-order polynomial occurs between two adjacent support points.

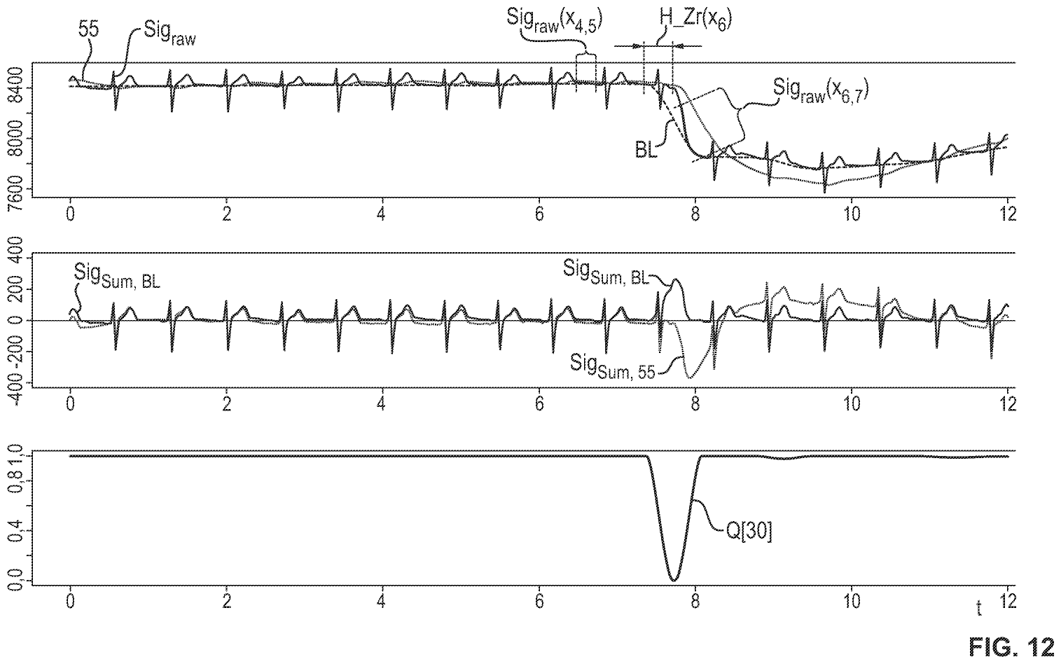

FIG. 12 shows an example of the raw signal Sig_{raw} from FIG. 7 and FIG. 9 and two average curves (zero lines) BL, 55 generated in different ways. The average curve BL was generated as described with reference to FIG. 11. The average curve 55 was generated by applying a low pass filter (not shown) to the raw Sig_{raw} signal. This low pass filter filters out high frequencies.

It is possible to apply the process explained with reference to FIG. 11 and FIG. 12 at least twice. In the first application, the raw signal Sig_{raw} is used as the input signal, and the first application provides as the output signal the sum signal Sig_{Sum,BL}. In the second application, the sum signal Sig_{Sum,BL} is used as the input signal, and the second application provides as the output signal a further smoothed sum signal Sig_{Sum,BL,BL}.

In addition, FIG. 12 shows the sum signal Sig_{Sum,BL}, which is generated when the average curve BL is generated as described with reference to FIG. 11. The following applies: Sig_{Sum,BL}=Sig_{raw}−BL. For comparison, the sum signal Sig_{Sum,55} is also shown, which is generated when the average curve 55 is used instead of the average curve BL. The following applies: Sig_{Sum,55}=Sig_{raw}−55. It can be seen that the sum signal Sig_{Sum,BL} only has an outlier for a single heartbeat x₆, while the sum signal Sig_{Sum,55} has an outlier for six heartbeats x₆ to x₁₁.

In addition, FIG. 12 shows the time course of the quality measure Q[30], which is generated when using the baseline BL.

In one embodiment, the average curve BL is treated as a random variable, preferably as a normally distributed random variable. Instead of an average curve, a curve depending on isoelectric points can also be calculated and used.

In the example shown, the measured value conditioner 19 comprises the following functional units, cf. FIG. 13:
A functional unit 40 detects the QRS segment in the raw signal Sig_{raw}, cf. FIG. 1.
A functional unit 41 detects the length of a currently evaluated section in the raw signal Sig_{raw}.
A functional unit 42 detects a support point in the currently evaluated section, for example as the 20% quantile.
A functional unit 43 constructs a spline for each heartbeat x by interpolation. The sequence of these splines acts as the average curve BL, which is subtracted from the raw signal Sig_{raw}. The spline can also be generated depending on isoelectric points.

The function block 30 includes the following functional units:
A functional unit 50 evaluates the regularity with which the functional unit 40 detects the QRS segments. If, in a specified evaluation period, the largest time interval between two QRS segments in immediate succession is at least twice as large as the smallest time interval, this is an indication that individual heartbeats have not been detected. This reduces the quality.
A functional unit 51 detects evaluation sections that are particularly long or particularly short. This reduces the quality.
A functional unit 52 determines the standard deviation of the random variable, which is the average curve BL. A large standard deviation is an indication of strongly fluctuating measured values and therefore reduces the quality.
A functional unit 53 evaluates the changes between the splines of two immediately successive heartbeats. A large change between two splines usually results from the fact that an offset of the signal has changed abruptly, which usually results from a measurement error, for example, because the contact between a measurement electrode and the skin of patient P has changed. The large deviation leads to a reduction in quality. For example, the quotient between the deviation of two consecutive splines on the one hand and the average magnitude (difference between the largest value and the smallest value) of a QRS segment on the other hand is used.
A functional unit 59 combines the individual quality measures calculated by functional units 50 through 53 into the quality measure Q[30].

The function block 30 provides a total quality measure Q[30].

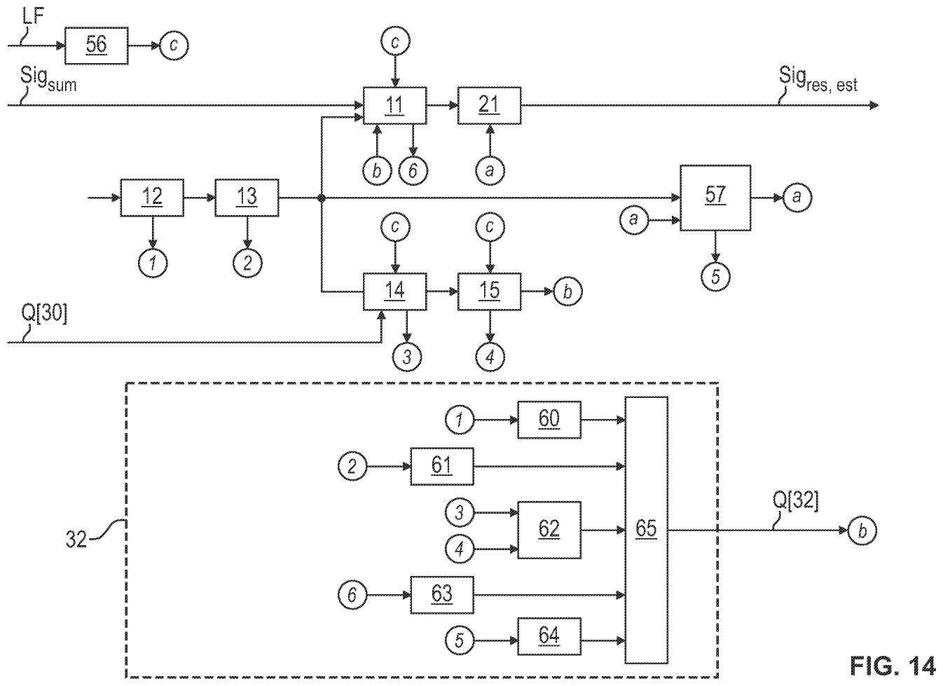

FIG. 14 shows in detail the function block 32. As already explained, the function block 32 evaluates whether the determined cardiogenic reference signal segment SigA_{kar,ref} or the adapted cardiogenic signal segment SigAₖₐᵣ(x) for a heartbeat x matches predefined expectations for a cardiogenic signal segment, cf. FIG. 8. The function block 32 provides a quality measure Q[32].

The following functional units are shown in FIG. 14:
As already mentioned, the functional unit 12 identifies the respective heartbeat period H_Zr(x) of each heartbeat x, preferably the QRS phase, in the sum signal Sig_{Sum}. The functional unit 13 identifies the respective characteristic heartbeat time H_Zp(x) of each heartbeat x.
As mentioned above, the functional unit 14 computationally superimposes the N sum signal segments SigA_{Sum}(x₁), . . . , SigA_{Sum}(x_{N}) positioned in the correct time for the last N heartbeats x₁, . . . , x_{N}. The functional unit 15 generates a cardiogenic reference signal segment SigA_{kar,ref} from the superposition of N sum signal segments SigA_{Sum}(x₁), . . . , SigA_{Sum}(x_{N}).
An optional functional unit 56 calculates a shape-changing factor for the cardiogenic reference signal segment SigA_{kar,ref} depending on a value of an anthropological parameter of the patient P during the heartbeat x and thereby generates an adapted cardiogenic signal segment SigAₖₐᵣ(x) for a heartbeat x. The anthropological parameter is described in WO 2021/063601 A1 (corresponding US2022330837 (A1) is discussed above) as an example. The anthropological parameter is, for example, the current lung filling level or the current position of the patient P. An exemplary mode of operation of this functional unit is described in WO 2021/063601 A1.
The functional unit 11 subtracts from the sum signal Sig_{Sum} the cardiogenic reference signal segment SigA_{kar,ref} or an adjusted cardiogenic signal segment SigAₖₐᵣ(x).
The optional attenuation function block 21 performs attenuation of the reference cardiogenic signal segment SigA_{kar,ref} or the matched cardiogenic signal segment SigAₖₐᵣ(x) to remove remaining segments of the cardiogenic signal Sigₖₐᵣ.
The optional functional unit 57 performs a residual power analysis and exchanges signals with the attenuation function block 21 for this purpose. In such a residual power analysis, the signal strengths of modifying signals Mod(i) are examined, and optionally attenuation is performed. Such a process is described, for example, in DE 10 2020 002 572 A1 (discussed above).

The functional units 12, 13, 16 and 21 perform the respective computation steps with a high sampling frequency of a few milliseconds so that the respective result is already available during the respective heartbeat. The functional units 14, 15 and 57 perform the computation steps with a lower sampling frequency and process the N sum signal segments SigA_{Sum}(x₁), . . . , SigA_{Sum}(x_{N}) of N already completed heartbeats.

Function block 32 comprises the following functional units:
A functional unit 60 evaluates the quality with which the functional unit 12 detects the heartbeat period H_Zr(x) of each heartbeat x.
A functional unit 61 evaluates the quality with which the functional unit 13 detects the exact heartbeat time point H_Zp(x) of each heartbeat x.
A functional unit 62 evaluates the quality with which a cardiogenic reference signal segment SigA_{kar,ref} or an adapted cardiogenic signal segment SigAₖₐᵣ(x) is generated for the heartbeat x. For this purpose, the functional unit 62 processes signals from the two functional units 14 and 15.
A functional unit 63 evaluates the quality with which the functional unit 16 has subtracted the cardiogenic reference signal segment SigA_{kar,ref} or the adjusted cardiogenic signal segment SigAₖₐᵣ(x) from the sum signal Sig_{Sum}.
A functional unit 64 evaluates the quality with which the functional unit 57 analyzed the residual power.
A functional unit 65 calculates the quality measure Q[32] from the five individual quality measures of the five functional units 60 to 64.

While specific embodiments of the invention have been shown and described in detail to illustrate the application of the principles of the invention, it will be understood that the invention may be embodied otherwise without departing from such principles.

List of reference signs

| | |
|---|---|
| 1 | Ventilator, artificially ventilates and/or monitors |
| | patient P, comprises the signal processing unit |
| | 5 according to the invention comprises |
| 2.1 | Intercostal (near heart) pair of measuring |
| | electrodes, includes measuring |
| | electrodes 2.1.1 and 2.1.2, provides measured |
| | values for the electrical sum signal Sig_{Sum}(1) |
| 2.1.1, | Measuring electrodes of the intercostal pair 2.1 |
| 2.1.2, | |
| 2.1 | Pair of measuring electrodes close |
| | to the diaphragm, includes measuring |
| | electrodes 2.2.1 and 2.2.2, provides measured |
| | values for the electrical sum signal Sig_{Sum}(2) |
| 2.2.1, | Measuring electrodes of the pair close to the |
| 2.2.2 | diaphragm 2.2 |
| 3 | Pneumatic sensor in front of the |
| | patient's mouth P, measures |
| | the volume flow Vol' and the airway pressure P_{aw} |
| 4 | Optical sensor with an image acquisition |
| | device and an image processing unit, |
| | measures the geometry of the patient's body P, |
| | from which a value of an anthropological |
| | parameter, for example the current lung |
| | filling level or the patient position, is derived |
| | computationally |
| 5 | Signal processing unit, comprises |
| | the function blocks 20 and 21, |
| | performs the steps of the process |
| | according to the invention, has at |
| | least temporary read access and write |
| | access to the data memory 9 |
| 6 | Probe in esophagus Sp, measures pneumatic |
| | pressure Pes in esophagus Sp |
| 7 | Cuff around one wrist of patient P, holds catheter 17, |
| | which invasively measures blood pressure over time |
| 8.1 | Sensor in the form of a finger clip on one of the |
| | patient's fingers P, non-invasively measures |
| | the degree of saturation of the blood with |
| | oxygen |
| 8.2 | Sensor in the form of a finger clip |
| | on another finger of patient P, |
| | non-invasively measures patient P's blood pressure. |
| 9 | Data memory to which the signal processing |
| | unit 5 has at least intermittent read |
| | access and write access and in which the |
| | cardiogenic reference signal segment |
| | SigA_{kar,ref} is stored |
| 10 | Functional unit of the compensation function |
| | block 20 that generates a synthetic |
| | cardiogenic signal Sig_{kar,syn}, which |
| | is an approximation (estimate) for, particularly a |
| | representation of the cardiogenic signal |
| | Sigₖₐᵣ and is composed of signal segments |
| 11 | Compensation function block 11 computationally |
| | compensates for the contribution of the |
| | cardiogenic signal Sigₖₐᵣ to m sum signal |
| | Sig_{Sum}, for example by subtracting the synthetic |
| | cardiogenic signal Sig_{kar,syn}, thereby |
| | generating the compensation signal Sig_{com} |
| 12 | Functional unit of the signal |
| | processing unit 5: detects |
| | the respective QRS time span (QRS segment) of |
| | each heartbeat in the sum signal Sig_{Sum} |
| 13 | Functional unit of the signal processing |
| | unit 5: detects the exact heartbeat |
| | time point H_Zp(x) of each heartbeat x |
| 14 | Functional unit of the compensation |
| | function block 20: superimposes |
| | computationally N sum signal segments |
| | SigA_{Sum}(x₁), . . . , SigA_{Sum}(x_{N}) for the last |
| | N heartbeats. |
| 15 | Functional unit of the compensation |
| | function block 20: generates a |
| | cardiogenic reference signal segment SigA_{kar,ref} |
| 16 | Functional unit of the compensation function |
| | block 20: positions the cardiogenic reference |
| | signal segments SigA_{kar,ref} or the adjusted |
| | cardiogenic signal segments SigAₖₐᵣ(x) at the |
| | correct time depending on the heartbeat |
| | time H_Zp(x), assembles the positioned |
| | cardiogenic reference signal segments SigA_{kar,ref} to |
| | the synthetic cardiogenic signal Sig_{kar,syn} |
| 17 | Catheter, held by cuff 7, invasively measures |
| | patient's blood pressure over time P |
| 19 | Measured value conditioner, generates the |
| | sum signal Sig_{Sum} from the measured values |
| | of the measuring electrodes 2.1.1 to 2.2.2. |
| 20 | Compensation function block: generates the |
| | synthetic cardiogenic signal Sig_{kar,syn} |
| | and the compensation signal Sig_{com} |
| 21 | Attenuation function block: generates the |
| | estimated respiratory signal Sig_{res,est} by attenuation |
| | from the compensation signal Sig_{com} |
| 22 | Function block, which determines |
| | the respective characteristic |
| | heartbeat time H_Zp(x) of each heartbeat x, |
| | comprises the functional units 12 and 13 |
| 30 | Function block: evaluates the quality |
| | with which the sum signal |
| | Sig_{Sum} was generated from the |
| | raw signal Sig_{raw}, provides the |
| | quality measure Q[30], includes the |
| | functional units 50 to 53 and 59 |
| 31 | Function block: evaluates with which |
| | reliability the characteristic |
| | heartbeat time point H_Zp(x) was |
| | detected by evaluating the sum |
| | signal Sig_{Sum}, provides the quality measure |
| | Q[31], includes the functional units |
| 32 | Function block: evaluates the quality with |
| | which the cardiogenic reference signal segment |
| | SigA_{kar,ref} or the determined cardiogenic |
| | signal segment SigA_{kar,syn}(x) |
| | was determined for a heartbeat x, |
| | provides the quality measure Q[32], comprises |
| | the functional units 60 to 65 |
| 40 | Functional unit: detects in the raw signal |
| | Sig_{raw} the QRS segment, |
| 41 | Functional unit: detects the length of a currently |
| | evaluated section in the raw signal Sig_{raw} |
| 42 | Functional unit: detects a support point in the |
| | currently evaluated section |
| 43 | Functional unit: constructs one spline for each |
| | heartbeat by interpolation |
| 50 | Functional Unit: evaluates the regularity with which |
| | the Functional Unit 40 detects the QRS segments. |
| 51 | Functional unit: detects evaluation sections that |
| | are particularly long or particularly short |
| 52 | Functional unit: determines the standard |
| | deviation of the random variable |
| 53 | Functional unit: evaluates the changes between the |
| | splines of two immediately consecutive heartbeats. |
| 55 | Average curve (zero line), which is |
| | generated by low-pass filtering |
| | from the raw signal Sig_{raw} |
| 56 | Functional unit: calculates a shape-changing |
| | factor for the cardiogenic reference |
| | signal segment SigA_{kar,ref} depending on the |
| | current lung filling level and thereby |
| | generates a cardiogenic signal |
| | segment SigAₖₐᵣ(x) for a heartbeat x. |
| 57 | Optional functional unit: analyzes the residual power |
| 59 | Functional unit: calculates the quality |
| | measure Q[30] from the individual quality |
| | measures of the functional units 50 to 53. |
| 60 | Functional unit: evaluates the quality with |
| | which the functional unit 12 detects the |
| | heartbeat period H_Zr(x) of each heartbeat x |
| 61 | Functional unit: evaluates the quality with which |
| | the functional unit 13 detects the exact |
| | heartbeat time H_Zp(x) of each heartbeat x |
| 62 | Functional unit: evaluates the quality with |
| | which a cardiogenic reference signal |
| | segment SigA_{kar,ref} or an adjusted cardiogenic |
| | signal segment SigAₖₐᵣ(x) is generated for heartbeat x |
| 63 | Functional unit: evaluates the quality with |
| | which the functional unit 16 subtracts the |
| | cardiogenic reference signal segment SigA_{kar,ref} or |
| | the adjusted cardiogenic signal segment SigAₖₐᵣ(x) |
| | from the sum signal Sig_{Sum} |
| 64 | Functional unit: evaluates the quality with which the |
| | functional unit 57 has analyzed the |
| | residual performance |
| 65 | Functional unit: calculates the quality measure Q[32]. |
| Atm(1), | Period for a patient's own breathing activity P |
| Atm(2), | |
| . . . | |
| BL | Average curve (baseline), generated as a spline over |
| | the interpolation points Stp(1, 2), Stp(2, 3), . . . |
| G | Matrix, each has a time course of the total quality |
| | measure Q for a heartbeat |
| H_Zp(x) | Characteristic heartbeat time point of the heartbeat |
| | x, detected by the functional unit 13 |
| H_Zp_{ref} | Reference heartbeat time |
| H_Zr(x) | Heartbeat time period of the heartbeat x, detected |
| | by the functional unit 12 |
| H_Zr_{ref} | Reference heartbeat period covered by cardiogenic |
| | reference signal segment SigA_{kar,ref}. |
| Ip | Initialization phase, includes N successive heartbeat |
| | periods H_Zr(x1), . . . , H_Zr(xN) |
| Irr_{kar,ref} | Deviating course due to disturbances as part of the |
| | cardiogenic reference signal segment SigA_{kar,ref} |
| N | Number of heartbeat periods of the |
| | initialization phase Ip |
| nc(G) | Matrix in which the sum of the values is |
| | normalized to one in each column |
| Np | Use phase in which the cardiogenic reference |
| | signal segment SigA_{kar,ref} and the detected |
| | heartbeat time points H_Zp(y1), . . . Are |
| | used to generate the compensation signal Sig _{com} |
| Q[30] | Quality measure for the quality with |
| | which the sum signal Sig_{Sum} was generated from |
| | the measured values of sensors 2.1.1 to 2.2.2, |
| | calculated by function block 30. |
| Q[31] | Quality measure for the reliability with which the |
| | characteristic heartbeat time H_Zp(x) was detected, |
| | calculated by function block 31. |
| Q[32] | Quality measure for the quality with which |
| | the cardiogenic reference signal segment |
| | SigA_{kar,ref} or the determined |
| | cardiogenic signal segment |
| | SigA_{kar,syn}(x) was determined for a heartbeat x, |
| | calculated by function block 32 |
| S | Matrix, has in each row a sum |
| | signal segment SigA_{Sum}(x), |
| | SigA_{Sum}(y) for one heartbeat x and y, respectively. |
| Sig_{com} | Compensation signal, is generated by the |
| | compensation function block 20 by |
| | compensating the contribution of the synthetic |
| | cardiogenic signal Sig_{kar,syn} to the sum signal Sig_{Sum} |
| Sigₖₐᵣ | Cardiogenic signal, causes the patient's |
| | heart activity P, estimated |
| | by the synthetic cardiogenic signal Sig_{kar,syn} |
| SigAₖₐᵣ(x) | Adjusted cardiogenic signal segment, |
| | refers to the heartbeat X, |
| SigA_{kar,ref} | Cardiogenic reference signal segment, describes |
| | approximately the course of the cardiogenic |
| | signal Sigₖₐᵣ during a single heartbeat, |
| | refers to the reference heartbeat period H_Zr_{ref} |
| Sig_{kar,syn} | Synthetic cardiogenic signal, is an estimate |
| | for the cardiogenic signal Sigₖₐᵣ, generated |
| | by the functional unit 10 from the signal |
| | segments SigA_{kar,syn}(x) |
| SigA_{kar,syn}(x) | Synthetic cardiogenic signal segment for |
| | heartbeat x, generated from the cardiogenic |
| | reference signal segment SigA_{kar,ref} using a |
| | value of an anthropological parameter |
| | measured at heartbeat x |
| Sig_{raw} | Raw signal from the measuring electrodes |
| | 2.1.1 to 2.2.2 |
| Sig_{raw}(x)_{1,2} | Section of the raw signal Sig_{raw} between the two |
| | heartbeat periods H_Zr(x₁) and H_Zr(x)₂ |
| Sigᵣₑₛ | Respiratory signal to be determined, correlates |
| | with the patient's own breathing activity P, |
| | which is the breathing activity caused by |
| | the diaphragm muscles |
| Sig_{res,est} | Estimate for the respiratory signal Sig |
| | to be determined.ᵣₑₛ |
| Sig_{Sum} | Sum signal, comprises a superposition of the |
| | respiratory signal Sigᵣₑₛ with the cardiogenic |
| | signal Sigₖₐᵣ, derived by the function |
| | block 19 from the raw signal Sig_{raw} |
| Sig_{Sum,BL} | Sum signal generated according to the computation |
| | rule Sig_{Sum,BL} = Sig_{raw}-BL |
| Sig_{Sum,55} | Sum signal, which is generated according to the |
| | computation rule Sig_{Sum,55} = Sig_{raw}-55 |
| SigA_{Sum}(x), | Sum signal segment for the heartbeat x or x₁, |
| SigA_{Sum}(x₁), | generated from the sum signal Sig_{Sum}, refers |
| | to the reference heartbeat period H_Zr_{ref} |
| Sp | Patient esophagus P |
| Stp(1,2) | Support point for the Sig_{raw}(x_{1,2}) |
| Zw | Diaphragm of the patient P |

What is claimed is:

1. A process for determining a cardiogenic reference signal segment, the segment describing cardiac activity of a patient in the course of a heartbeat, the process comprising the steps of:
providing a sensor arrangement that measures at least one variable in a patient's body or at the patient's body
generating a sum signal by pre-processing measured values of the sensor arrangement, the generated sum signal comprising a superposition of a respiratory signal, which describes the patient's own breathing activity, with a cardiogenic signal, which describes the patient's cardiac activity;
generating a sample which comprises a respective sum signal segment for each heartbeat of a sample sequence of heartbeats where the sum signal segment for a heartbeat of the sample sequence describes a course of the sum signal in the course of the respective heartbeat;
for each heartbeat of the sample sequence detecting a respective characteristic heartbeat time point; and
determining the cardiogenic reference signal segment using an aggregation of the sum signal segments of the generated sample and the characteristic heartbeat time points of the sample sequence,
wherein the aggregation is determined using at least one respective weight factor for each sum signal segment,
wherein the respective weight factor for the sum signal segment of a heartbeat of the sample sequence is determined depending on at least one of the following quality measures:
a quality measure for a quality with which the sum signal and/or the sum signal segment for the related heartbeat have been generated by pre-processing the measured values,
a quality measure for a reliability with which the characteristic heartbeat time point of the related heartbeat has been detected, and
a quality measure that assesses a shape of the sum signal segment or that assesses a shape of the generated cardiogenic reference signal segment, and
wherein the weight factor or each weight factor is the greater the greater the quality measure used or the greater each quality measure used.

2. A process according to claim 1, further comprising the step of approximately determining the respiratory signal, wherein the contribution of the cardiogenic signal to the sum signal is at least partially compensated by computation using the cardiogenic reference signal segment.

3. A process according to claim 2, wherein the step of computationally compensating the contribution of the cardiogenic signal comprises the steps of:
detecting the respective characteristic heartbeat time point for each heartbeat of a heartbeat sequence of heartbeats;
duplicating for each heartbeat of the heartbeat sequence the cardiogenic reference signal segment and temporally positioning the duplicates in relation to the sum signal using the respective characteristic heartbeat time point for that heartbeat; and
determining a difference between the sum signal and the positioned cardiogenic reference signal segments of the heartbeat sequence.

4. A process according to claim 1, further comprising the step of approximately determining the cardiogenic signal, wherein the determination of the cardiogenic signal comprises the steps of:
detecting the respective characteristic heartbeat time point for a heartbeat sequence of heartbeats; and
duplicating for each heartbeat of the heartbeat sequence the cardiogenic reference signal segment and combining the duplicates by using the determined characteristic heartbeat time points to form the cardiogenic signal.

5. A process according to claim 1, wherein:
the sample comprises N sum signal segments for N heartbeats, where N>1 is a predetermined number;
the step of determining the cardiogenic reference signal segment is performed repeatedly; and
for each execution of the determination, a sample with N sum signal segments is generated and used for the respective most recent completely executed N heartbeats.

6. A process according to claim 5, wherein:
a first cardiogenic reference signal segment is determined with an initial determination; and
each subsequent determination of the cardiogenic reference signal segment is based on a sample with N sum signal segments and uses the cardiogenic reference signal segment determined during the previous determination.

7. A process according to claim 1, wherein for at least one heartbeat of the sample sequence a value is measured wherein an anthropological parameter for the patient assumes in the course of the heartbeat this value, and an adjusted cardiogenic signal segment for the heartbeat is generated from the generated cardiogenic reference signal segment using the value of the anthropological parameter measured for the heartbeat.

8. A signal processing unit for generating a cardiogenic reference signal segment, the segment describing the cardiac activity of a patient in the course of a heartbeat,
wherein the signal processing unit is configured to receive measured values of a sensor arrangement, wherein the sensor arrangement is configured to measure at least one variable in a body of the patient or at the body of the patient,
wherein the signal processing unit is configured to generate a sum signal by pre-processing measured values of the sensor arrangement, wherein the sum signal comprises a superposition of a respiratory signal, describing the patient's own breathing activity, with a cardiogenic signal, describing the patient's cardiac activity,
wherein the signal processing unit is configured to generate a sample, wherein the generated sample comprises, for each heartbeat of a sample sequence of heartbeats, a respective sum signal segment, wherein the sum signal segment for a heartbeat of the sample sequence describes a course of the sum signal in the course of the related heartbeat of the sample sequence,
wherein the signal processing unit is configured to detect a respective characteristic heartbeat time point for each heartbeat of the sample sequence and to determine a cardiogenic reference signal segment using an aggregation of the sum signal segments of the generated sample and the characteristic heartbeat time points of the sample sequence,
wherein the signal processing unit is configured to perform the aggregation using at least one weight factor for each sum signal segment and the characteristic heartbeat time points of the sample sequence,
wherein the signal processing unit is configured to determine the respective weight factor for the sum signal segment of a heartbeat of the heartbeat sample depending on at least one of the following quality measures:
a quality measure for a quality with which the sum signal and/or the sum signal segment for the related heartbeat have been generated by the measured value pre-processing;
a quality measure for a reliability with which the characteristic heartbeat time point of the related heartbeat have been detected; and
a quality measure that assesses a shape of the sum signal segment or that assesses a shape of the generated cardiogenic reference signal segment, and
wherein the at least one weight factor is the greater the greater quality measure used or the greater each quality measure used.

9. A signal processing unit according to claim 8, wherein the signal processing unit is configured, for at least one heartbeat of the sample sequence,
to receive a measured value, an anthropological parameter assuming the measured value for the patient in the course of the at least one heartbeat of the sample sequence, and
to generate an adjusted cardiogenic signal segment for the at least one heartbeat from the generated cardiogenic reference signal segment using the value of the anthropological parameter measured for this heartbeat.

10. A system comprising:
a sensor arrangement configured to measure at least one variable in a patient's body or at the patient's body and provide measured values; and
a signal processing unit for generating a cardiogenic reference signal segment, the segment describing the cardiac activity of a patient in the course of a heartbeat, the signal processing unit comprising:
a sum signal generator configured to generate a sum signal from the measured values of the sensor arrangement by pre-processing measured values, the generated sum signal comprising a superposition of a respiratory signal, describing the patient's own breathing activity, with a cardiogenic signal, describing the patient's cardiac activity;
a sum signal segment generator generating a sum signal segment for each heartbeat of a sequence of heartbeats where the respective sum signal segment for a heartbeat of the sequence describes a course of the sum signal in the course of the respective heartbeat;
a characteristic heartbeat time point functional unit for detecting for each heartbeat of the sequence of heartbeats a respective characteristic heartbeat time point;
a cardiogenic reference signal segment functional unit for determining the cardiogenic reference signal segment using an aggregation of the sum signal segments and the characteristic heartbeat time points of the sequence of heartbeats, wherein the aggregation is determined using for each sum signal segment at least one respective weight factor; and
a weight factor determination means configured to determine the respective weight factor for the sum signal segment of a heartbeat of the heartbeat sample depending on at least one of the following quality measures:
a quality measure for a quality with which the sum signal and/or the sum signal segment for the related heartbeat have been generated by the measured value pre-processing;
a quality measure for a reliability with which the characteristic heartbeat time points of the heartbeats have been detected; and
a quality measure that assesses a shape of the sum signal segment or that assesses a shape of the generated cardiogenic reference signal segment, and
wherein the at least one weight factor is the greater the greater the at least one quality measure used or the greater each quality measure used.

11. A system according to claim 10, wherein the signal processing unit further comprises a compensation function block to determine a representation of the respiratory signal, wherein the contribution of the cardiogenic signal to the sum signal is at least partially compensated by computation using the cardiogenic reference signal segment.

12. A system according to claim 11, wherein the processing unit computationally compensates the contribution of the cardiogenic signal by:
detecting the respective characteristic heartbeat time point for each heartbeat of a heartbeat sequence of heartbeats;
duplicating for each heartbeat of the heartbeat sequence the cardiogenic reference signal segment and temporally positioning the duplicates in relation to the sum signal using the respective characteristic heartbeat time points for that heartbeat; and
determining a difference between the sum signal and the positioned cardiogenic reference signal segments of the heartbeat sequence.

13. A system according to claim 10, wherein the processing unit determines a representation of the cardiogenic signal based on the characteristic heartbeat time points for the sequence of heartbeats and a combination of the cardiogenic reference signal segments and the determined characteristic heartbeat time points.

14. A system according to claim 10, wherein with the processing unit:
N sum signal segments are provided for N heartbeats, where N>1 is a predetermined number;
the cardiogenic reference signal segment is determined repeatedly; and
for each determination, the N sum signal segments are generated and used for the respective most recent N heartbeats.

15. A system according to claim 14, wherein with the processing unit:
a first cardiogenic reference signal segment is determined with an initial determination; and
each subsequent determination of the cardiogenic reference signal segment is based on N sum signal segments and uses the cardiogenic reference signal segment determined during the previous determination.

16. A system according to claim 10, wherein the signal processing unit is configured, for at least one heartbeat of the sample sequence,
to receive a measured value, an anthropological parameter assuming the measured value for the patient in the course of the at least one heartbeat of the sample sequence, and
to generate an adjusted cardiogenic signal segment for the at least one heartbeat from the generated cardiogenic reference signal segment using the value of the anthropological parameter measured for this heartbeat.
